(19) United States
(12) Patent Application Publication
(10) Pub. No.: US 2018/0098936 A1
(43) Pub. Date: Apr. 12, 2018

(54) METHODS AND COMPOSITIONS FOR INJECTING HIGH CONCENTRATION AND/OR HIGH VISCOSITY ACTIVE AGENT SOLUTIONS

(71) Applicant: PRESIDENT AND FELLOWS OF HARVARD COLLEGE, Cambridge, MA (US)

(72) Inventors: Donald E. INGBER, Boston, MA (US); David A. WEITZ, Boston, MA (US); Jonathan DIDIER, Cambridge, MA (US); Maximilian ZIERINGER, Cambridge, MA (US)

(73) Assignee: PRESIDENT AND FELLOWS OF HARVARD COLLEGE, Cambridge, MA (US)

(21) Appl. No.: 15/560,653

(22) PCT Filed: Mar. 23, 2016
(86) PCT No.: PCT/US16/23744

§ 371 Date: Sep. 22, 2017

Related U.S. Application Data
(60) Provisional application No. 62/136,954, filed on Mar. 23, 2015.
(60) Provisional application No. 62/175,528, filed on Jun. 15, 2015.

(51) Int. Cl. A61K 9/00; A61K 9/113; A61K 9/50; A61K 47/32; A61K 47/26; A61K 47/44; A61K 47/34; A61K 47/06

(52) U.S. Cl. CPC A61K 9/0019; A61K 9/113; A61K 9/5089; A61K 47/06; A61K 47/26; A61K 47/44; A61K 47/34; A61K 47/32

(57) ABSTRACT

Embodiments of various aspects described herein relate to methods and compositions for injecting and/or delivering high viscosity and/or high concentration active agent solutions. In some embodiments, the methods and compositions described herein can be used for subcutaneous administration.

### RELATED APPLICATIONS

This application claims benefit under 35 U.S.C. § 119(e) of the U.S. Provisional Application No. 62/136,954, filed Mar. 23, 2015 and No. 62/175,528, filed Jun. 15, 2015 contents of both of which are incorporated herein by reference in their entireties.

### TECHNICAL DISCLOSURE

Embodiments of various aspects described herein relate to methods, compositions, and devices for injecting high viscosity and/or high concentration active agent solutions.

### BACKGROUND

In recent years, pharmaceutical companies have turned increasingly to high-concentration biologics or protein formulations. Such drug formulations can offer patients the convenience of subcutaneous injection, e.g., in a clinic or even at home—instead of a trip to a hospital for an intravenous infusion treatment. However, a subcutaneous formulation has to deliver the same dose of medicine in a 1-mL or 2-mL syringe that is delivered by a much higher volume of 250 mL or more in intravenous infusion, while intravenous formulations can be lower in concentration. Therefore, high-concentration therapeutic formulations are desirable.

Yet, high-concentration therapeutic formulations present a problem. For example, an increase in the concentration of biologics or proteins (e.g., antibodies, vaccine components, and/or enzymes) results in a nonlinear increase in viscosity, which rapidly presents a limitation to the subcutaneous administration with conventional means (e.g., syringes). Specifically, highly viscous preparations of therapeutic agents develop a high back-pressure and therefore compromise proper injection and/or infusion. In particular, a prolonged duration of administration compared to preparations having a lower concentration can be expected. While increasing needle size can reduce injection time, using a larger needle can increase pain and decrease its usefulness. These drawbacks can consequently lead to a decrease in the acceptance of the subcutaneous route. Further, with regard to the manufacturing process, the handling of a highly viscous preparation of therapeutic agents is relatively cumbersome. Accordingly, there is a need for developing new methods and compositions for injecting or delivering high viscosity and/or high concentration therapeutic formulations.

### SUMMARY

Embodiments of various aspects described herein relate to compositions, emulsions, devices, and methods for injecting or delivering a high viscosity and/or high concentration active agent solution. Examples of an active agent suitable to be administered using various aspects described herein include, but are not limited to, a protein, a peptide, an antibody, a growth factor, a nucleic acid, a sugar, an antigen, a vaccine, an enzyme, a cell, a small molecule covalently linked to a polymer, and a combination of two or more thereof. In particular, the compositions, emulsions, devices, and methods described herein are, in part, based on forming an emulsion in which liquid-based droplets comprising an active agent at a high concentration and/or high viscosity are distributed in a lower viscosity injection solution. Thus, the required dose can be delivered subcutaneously through a small gauge needle (e.g., equal to or greater than 18 gauge, or about 25-30 gauge) in a typical injection volume (e.g., less than 1.5 mL). In some embodiments, the droplets can comprise an outer shell or a solidified shell encapsulating a high viscosity of an active agent solution. These droplets can also confer additional stability to the encapsulated active agents such as drugs or bioactives. For example, the outer shell or solidified shell can protect the encapsulated active agent solution from external influences or environmental influences, including, e.g., light, change in pH, change in salinity or osmotic effects, and humidity, during administration, transport, handling, and/or storage. The outer shell or solidified shell can also be designed to allow for sustained release of the inner phase over a period of time, e.g., over hours, days, weeks or months. The outer shell or solidified shell can also be designed to allow for the administration into particular compartments of the body, e.g., the eye.

Accordingly, one aspect described herein relates to a composition comprising: (i) droplets comprising a first liquid, the first liquid comprising an active agent at a concentration of at least about 50 mg/mL or higher; and (ii) a carrier liquid. The carrier liquid and the droplets are substantially immiscible, and the droplets are dispersed in the carrier liquid.

In some embodiments, the concentration of the active agent can be at least about 100 mg/mL or higher.

In some embodiments, the first liquid comprising an active agent can have a viscosity of about 0.8 cP to about 500 cP. In some embodiments, the first liquid comprising an active agent can have a viscosity of about 20 cP to about 500 cP. In some embodiments, the first liquid comprising an active agent can have a viscosity of about 200 cP to about 500 cP. The viscosity measurements are generally measured at room temperature or at a temperature of about 25° C.

Another aspect described herein is a composition comprising: (i) droplets comprising a first liquid, the first liquid comprising an active agent and having a viscosity of at least about 20 cP or higher; and (ii) a carrier liquid. The carrier liquid and the droplets are substantially immiscible, and the droplets are dispersed in the carrier liquid. In some embodiments, the active agent-comprising first liquid can have a viscosity of at least about 50 cP or higher. In some embodiments, the active agent can be present in the first liquid at a concentration of at least about 50 mg/mL or higher.

In some embodiments of this aspect and other aspects described herein, the carrier liquid can have a lower viscosity than that of the active agent-comprising first liquid. In some embodiments, the viscosity of the carrier liquid can be selected such that it can be injected properly (e.g., using a syringe with a small needle). For example, the viscosity of the carrier liquid can have a range of about 0.5 cP to less than 500 cP, e.g., when measured at room temperature or at a temperature of about 25° C. In one embodiment, the viscosity of the carrier liquid can be no more than 450 cP. In one embodiment, the viscosity of the carrier liquid can be no more than 200 cP. The viscosity of the carrier liquid can vary with the needle size. By way of example only, in one embodiment for injection (e.g., subcutaneous injection) through a 25-27 gauge needle, the viscosity of the carrier liquid should be no more than 50 cP. However, for some injections where a 16-17 gauge needle can be acceptable (though it may cause some pain), the carrier liquid can have a much higher viscosity.

While the droplets can be of any dimension provided that they can be delivered in the form of an emulsion, in some embodiments of this aspect and other aspects described herein, the droplets can be microdroplets. In some embodiments, the microdroplets can have a size of about 1 μm to about 500 μm in diameter.

In some embodiments, the composition, the first liquid, and/or the carrier liquid can further comprise an additive. An additive can be selected to reduce or minimize aggregation and/or denaturation of an active agent to be encapsulated in the droplets, and/or to stabilize the dispersion of droplets in a carrier liquid. Accordingly, an additive can include, but is not limited to, a stabilizer, a surfactant, a buffered solution, and/or a polymer. Non-limiting examples of a surfactant include polyvinylalcohol, Span 80, Tween 80, sodium dodecyl sulfate, or a combination of two or more thereof. Polymer(s) added into the composition, the first liquid, and/or the carrier liquid as an additive can be any water-soluble polymer described herein or oil-soluble polymer described herein. Examples of a water-soluble polymer to be added as an additive include, but are not limited to, PEG-based polymer, dextran, or a combination of both. In some embodiments, the polymer(s) added into the composition, the first liquid, and/or the carrier liquid as an additive can include hydrogel. Exemplary hydrogels include, but are not limited to, alginate, gelatin, guar, PEG-based hydrogels, or a combination of two or more thereof.

The droplets in the compositions described herein can have at least one or more (e.g., at least two or more) liquid phases. In some embodiments, the droplets can be of a single liquid phase. In these embodiments, the droplets can be formed from a first liquid comprising an active agent as described herein. In some embodiments, the first liquid can be an aqueous-based liquid, and the carrier liquid can be optionally an oil-based liquid. In some embodiments, the first liquid can be an oil-based liquid, and the carrier liquid can be optionally an aqueous-based liquid. In some embodiments, the first liquid can be an aqueous-based liquid comprising a first water-soluble polymer, and the carrier liquid can be an aqueous-based liquid comprising a second water-soluble polymer. In these embodiments, the first water-soluble polymer and the second water-soluble polymer are incompatible to each other. Examples of different combinations of the first water-soluble polymer and the second water-soluble polymer that can be used in the first liquid and carrier liquid, respectively, include, but are not limited to, polyacrylamide & poly(acrylic acid); polyacrylamide & poly(methacrylic acid); polyacrylamide & poly(ethylene glycol) (PEG); polyacrylamide & poly(2-ethyl-2-oxazoline); polyacrylamide & polyethylenimine (PEI); polyacrylamide & polyvinyl alcohol (PVA); polyacrylamide & Pluronic F68; polyacrylamide & Triton X-100; polyacrylamide & Tween 20; polyacrylamide & poly(propylene glycol); polyacrylamide & N;N-dimethyldodecylamine N-oxide; polyacrylamide & Zonyl; Ficoll & poly(methacrylic acid); Ficoll & dextran; Ficoll & PEG; Ficoll & poly(2-ethyl-2-oxazoline); Ficoll & PEI; Ficoll & PVA; Ficoll & hydroxyethyl cellulose; Ficoll & Pluronic F68; Ficoll & Triton X-100; Ficoll & Tween 20; Ficoll & Brij 35; Ficoll & methyl cellulose; dextran & Ficoll; dextran & poly(2-ethyl-2-oxazoline); dextran & PEG; dextran & PVA; hydroxyethyl cellulose; dextran & Pluronic F68; dextran & Triton X-100; dextran & Tween 20; dextran & poly(propylene glycol); dextran & polyvinylpyrrolidone (PVP); dextran & Zonyl; poly(acrylic acid) & polyacrylamide; poly(acrylic acid) & PEG; poly(acrylic acid) & alginic acid; poly(acrylic acid) & sodium dodecyl sulfate-polymer (SDS); poly(acrylic acid) & diethylaminoethyl-dextran; poly(methacrylic acid) & polyacrylamide; poly(methacrylic acid) & Ficoll; poly(methacrylic acid) & PEG; poly(methacrylic acid) & poly(2-ethyl-2-oxazoline); poly(methacrylic acid) & PEI; poly(methacrylic acid) & Pluronic F68; poly(methacrylic acid) & Triton X-100; poly(methacrylic acid) & Tween 20; poly(methacrylic acid) & carboxy-polyacrylamide; poly(methacrylic acid) & poly(propylene glycol); poly(methacrylic acid) & PVP; poly(methacrylic acid) & N;N-dimethyldodecylamine N-oxide; poly(methacrylic acid) & Zonyl; PEG & polyacrylamide; PEG & Ficoll; PEG & dextran; PEG & poly(acrylic acid); PEG & poly(methacrylic acid); PEG & PEI; PEG & PVA; PEG & poly(2-ethyl-2-oxazoline); PEG & Tween 20; PEG & dextran sulfate; PEG & PVP; PEI & polyacrylamide; PEI & Ficoll; PEI & poly(methacryilc acid); PEI & PEG; PEI & poly(2-ethyl-2-oxazoline); PEI & Pluronic F68; PEI & carboxy-polyacrylamide; PVA & polyacrylamide; PVA & Ficoll; PVA & dextran; PVA & PEG; PVA & PEI; PVA & Pluronic F68; PVA & Tween 20; PVA & dextran sulfate; PVA & carboxy-polyacrylamide; hydroxyethyl cellulose & Ficoll; hydroxyethyl cellulose & dextran; hydroxyethyl cellulose & Triton X-100; and hydroxyethyl cellulose & Tween 20.

In some embodiments, the droplets can each exhibit at least two immiscible liquid phases or more, and the two or more liquid phases within the droplets can be arranged in various configurations. For example, in some embodiments, the droplets can each comprise a first liquid and a second liquid, wherein the first liquid and the second liquid are immiscible. In some embodiments, the droplets can each comprise a core and a shell surrounding the core. For example, the first liquid can form the core of the droplets while the second liquid can form the shell surrounding the core, or vice versa. In alternative embodiments, the droplets can each comprise subdroplets dispersed therein. For example, the first liquid can form subdroplets dispersed in the second liquid that form the droplets, or vice versa.

Droplets of different combinations of immiscible liquids can be formed. In some embodiments, the first liquid can be an aqueous-based liquid, the second liquid can be an oil-based liquid, and the carrier liquid can be an aqueous-based liquid. In some embodiments, the first liquid can be an oil-based liquid, the second liquid can be an aqueous-based liquid, and the carrier liquid can be an oil-based liquid.

In some embodiments, at least two liquid phases can comprise a core of the first liquid and a shell of the second liquid surrounding the core. In some embodiments, the second liquid can be solidified to form a capsule or microcapsule. In some embodiments, the first liquid can comprise a preformed emulsion, in which an aqueous phase comprising the active agent is dispersed in an organic solvent. In these embodiments, the volume ratio of the aqueous phase to the organic solvent can range from about 1:5 to about 5:1. In alternative embodiments, the first liquid can comprise a preformed dispersion, in which solid particles comprising the active agent are dispersed in an organic phase or an aqueous phase. In these embodiments, the volume fraction of the solid particles within the dispersion can range from about 0.1 to about 0.75. In some embodiments, the solid particles can have a size of about 20 nm to about 5 μm. Examples of the organic phase used in the capsules described herein can include, but are not limited to perfluorohexane, dichloromethane, ethanol, ethyl acetate, dimethyl sulfoxide, and a combination of two or more there.

The droplets and the carrier liquid can constitute the compositions described herein in any volume ratio provided that there is sufficient volume of the carrier liquid to facilitate delivery of the droplets to a target site or area, e.g., via a small orifice such as an injection needle. Generally, the higher the volume ratio of the droplets to the carrier liquid, the higher amount of an active agent can be present in the compositions. In some embodiments, the droplets and the carrier liquid can be in a volume ratio of about 10:90 to about 75:25.

In some embodiments of various aspects described herein, an oil-based liquid, e.g., as part of a liquid phase within the droplets and/or the carrier liquid can include, but is not limited to, ethyl acetate, omega-3 oil, fish oil, silicone oil, parenteral oil, mineral oil, paraffin, fatty esters, olive oil, dichloromethane, sunflower oil, fluorinated oil, perfluoro alkane, and a combination of two or more thereof. In some embodiments, the oil-based liquid can further comprise an oil-soluble additive. In one embodiment, the oil-soluble additive can comprise an oil-soluble polymer. Exemplary oil-soluble polymers can include, without limitations, the oil-soluble polymer comprises polycaprolactone (PCL), poly (N-vinyl pyrolidone), poly glycolic acid (PGA), poly lactic-co-glycolic acid (PLGA), poly L-lactic acid (PLLA), poly propylene fumarate (PPF), polybutadiene, polyisoprene, and a combination of two or more thereof.

In some embodiments of various aspects described herein, an aqueous-based liquid, e.g., as part of a liquid phase within the droplets and/or the carrier liquid can include, but is not limited to, a buffered solution. The aqueous-based liquid can further comprise a water-soluble additive. In one embodiment, the water-soluble additive can comprise a water-soluble polymer. Non-limiting examples of a water-soluble polymer can include, but are not limited to, cellulose, dextran, poly acrylic acid (PAA), poly(ethylene glycol) (PEG), poly(vinyl acetate), polyvinyl alcohol (PVA), poly(lactic acid)(PLA), polyhydroxy ethyl methacrylate, polyacrylamide, polyethylene oxide, alginate, Polyacrylamide, poly(acrylic acid), poly(methacrylic acid), poly(2-ethyl-2-oxazoline), polyethylenimine (PEI), Pluronic F68, Triton X-100, Tween 20, poly(propylene glycol), N,N-dimethyldodecylamine N-oxide, Zonyl, Ficoll, dextran, poly(2-ethyl-2-oxazoline), hydroxyethyl cellulose, Brij 35, methyl cellulose, polyvinylpyrrolidone (PVP), Zonyl, poly(acrylic acid), alginic acid, sodium dodecyl sulfate-polymer (SDS), diethylaminoethyl-dextran, poly(methacrylic acid), carboxy-polyacrylamide, dextran sulfate, carboxy-polyacrylamide, and a combination of two or more thereof.

In some embodiments of this aspect and other aspects described herein, the composition does not include a viscosity-reducing agent. Exemplary viscosity-reducing agent is proline.

Injection of high-concentration therapeutic agent solution can interfere with the plunging action of syringes, as the high-concentration therapeutic agent solution generally tends to be viscous. The compositions described herein do not have such injection problem, because the high-concentration and/or high-viscosity therapeutics are formed into liquid droplets, which are then dispersed in a carrier liquid of a lower viscosity. Accordingly, methods for producing an injectable composition comprising a high-concentration and/or high-viscosity dose of active agent(s) are provided herein. In some embodiments, the volume of the injectable composition for a single dose of active agent(s) can be no more than 5 mL or no more than 1.5 mL. In some embodiments, the injectable composition can be characterized such that pressure for injection is no more than 125 mPa or lower.

In one aspect, the method of producing an injectable composition comprising a high-concentration dose of an active agent solution comprises: forming an emulsion comprising droplets dispersed in an injectable carrier liquid, wherein: the droplets comprise a first liquid, the first liquid comprising an active agent at a concentration of at least about 50 mg/mL; and the droplets and the injectable carrier liquid are substantially immiscible. Thus, an injectable composition comprising an emulsion with a high concentration dose of one or more active agent(s) can be produced.

In another aspect, described herein is a method of producing an injectable composition comprising a high-viscosity dose of active agent(s). The method comprises: forming an emulsion comprising droplets dispersed in an injectable carrier liquid, wherein: the droplets comprise a first liquid, the first liquid comprising an active agent and having a viscosity of at least about 20 cP; and the droplets and the injectable carrier liquid are substantially immiscible. In some embodiments, the viscosity of the active agent-comprising first liquid can be at least about 50 cP. Thus, an injectable composition comprising an emulsion with a high viscosity dose of one or more active agent(s) can be produced.

In some embodiments of this aspect and other aspects described herein, the droplets can be microdroplets.

In some embodiments of this aspect and other aspects described herein, the emulsion can be a single emulsion.

In some embodiments of this aspect and other aspects described herein, the emulsion can be a double or higher-order multiple emulsion.

Methods for forming different types of emulsions are known in the art and can be used to produce the compositions and emulsions described herein. In some embodiments, the emulsion can be formed using microfluidic technology, e.g., in a microfluidic channel. By way of example only, microfluidic droplet fabrication methods can be used to form droplets of a high viscosity active agent aqueous solution in a biocompatible carrier liquid (e.g., an oil-based liquid).

Depending on the size of the droplets formed in an emulsion, in some embodiments, the methods of various aspects described herein can further comprise reducing the droplets into smaller droplets. For example, in one embodiment, the droplets can be reduced into microdroplets.

If necessary, in some embodiments, the methods of various aspects described herein can further comprise converting a polydisperse population of droplets to a substantially monodisperse population of droplets.

If desirable, the droplets in an emulsion can be transferred to a different carrier liquid and/or concentrated in either the same or a different biocompatible carrier liquid. For example, the droplets in an emulsion can be concentrated to increase the number of droplets in a certain volume of a carrier liquid; thus, increasing the total amount of an active agent present in a certain volume of the carrier liquid.

Accordingly, in some embodiments, the methods of various aspects described herein can further comprise separating the droplets from the emulsion. The droplets can be separated from the emulsion or carrier liquid using any methods known in the art without causing droplet breakage. For example, the separating can be performed by centrifugation, size exclusion, filtration, and a combination of two or more thereof.

In some embodiments, the methods of various aspects described herein can further comprise re-dispersed the separated droplets in the same or a different carrier liquid. When the separated droplets are re-dispersed in a smaller volume of a carrier liquid, a more concentrated emulsion can be produced.

Emulsions or compositions produced by the methods of making high-concentration and/or high-viscosity injectable compositions are also described herein.

The compositions described herein (including emulsions and injectable compositions) can be packaged in a container or device. In some embodiments, the compositions described herein can be packaged in a vial, e.g., for injection. Accordingly, another aspect provided herein is a vial comprising one or more embodiments of the compositions, emulsions or injectable compositions described herein.

In some embodiments, the compositions described herein can be loaded into an injection device. Accordingly, described herein is also an injection device comprising (i) a chamber and (ii) one or more embodiments of the compositions, emulsions, or injectable compositions disposed in the chamber.

In some embodiments, the injection device can further comprise a needle adaptably coupled to the chamber. The needle can have a gauge of at least 18 or above. In some embodiments, the needle can have a gauge of about 25-30. In some embodiments, the needle can have a gauge of less than 18. In these embodiments, the droplets of the compositions, emulsions, or injectable compositions described herein can have a dimension that is smaller than the inner diameter of the needle. Thus, the compositions, emulsions, or injectable compositions can be injected quickly, while an active agent at a high concentration can still be delivered in a rapid manner.

In some embodiments, the injection device can be an autoinjector. In some embodiments, the injection device can be a prefilled syringe. In some embodiments, the chamber can comprise a syringe barrel.

In one aspect, the compositions, emulsions, and injection devices described herein are useful to administer a high concentration dose of an active agent to a subject in need thereof. Thus, methods of administering to a subject a high concentration dose of an active agent are also provided herein. The method comprises injecting the subject with one or more embodiments of the compositions or emulsions described herein, or using one or more embodiments of the injection devices described herein.

While the compositions, emulsions, and injection devices described herein can be used for any parenteral administration of an active agent, they can be more beneficial when used for subcutaneous administration where the injection volume is usually small, e.g., no more than 5 mL or no more than 1.5 mL. Thus, in some embodiments, the injection can be performed by subcutaneous administration. In some embodiments, the injection volume of the high-concentration dose can be no more than 5 mL or no more than 1.5 mL.

### BRIEF DESCRIPTION OF THE DRAWINGS

FIG. 1 is a schematic diagram showing an exemplary embodiment of a microfluidic device for fabrication of the compositions and/or emulsions described herein. The reservoir/channel for the inner phase is indicated with (1), the channels for the outer phase with (2) and the connecting channels with (3).

FIG. 2 is a photograph showing generation of double emulsion droplets at nozzles in a microfluidic Millipede single emulsion device, e.g., as shown in FIG. 1.

FIGS. 3A-3B show increasing viscosities as the concentration (FIG. 3A) or volume fraction (FIG. 3B) of the bioactive solution increases.

FIG. 4 is a flow diagram showing microencapsulation of a high viscosity/high concentration active agent solution according to one embodiment of the methods described herein. Encapsulation not only permits injection of high viscosity/high concentration active solution, but also protects the active agent from external influences during storage, handling, and/or injection. A pre-emulsion of a high viscosity/high concentration active agent solution dispersed in an evaporable organic phase is formed. The pre-emulsion is then subjected to microfluidic encapsulation such that the high viscosity/high concentration active agent solution is encapsulated within a polymer membrane. In some embodiments, the polymer membrane can be solidified, e.g., by solvent evaporation, thereby forming microcapsules comprising a core and a polymer shell with the active agent solution encapsulated in the core. The polymer membrane can provide additional stability of the active agent solution. The microcapsules can be dispersed in a low viscosity carrier fluid up to ˜70% v/v. In addition, encapsulation of a high viscosity/high concentration active agent solution in microcapsules can permit controlled and/or sustained release of the active agent if desired. The microencapsulation method described herein can have 100% encapsulation efficiency, permit precise control of droplet/capsule size, and/or precise control of the core-shell ratio of the capsules.

FIGS. 5A-5B show an exemplary process and/or device for fabrication of the compositions and/or emulsions described herein according to one embodiment described herein. FIG. 5A is a schematic diagram of a process and/or a device for fabrication of the compositions and/or emulsions described herein according to one embodiment described herein. An inner fluid is introduced through the injection tube disposed at a first end of an outer tube, while a middle fluid is introduced into the outer tube from the first end. Large droplets of the inner fluid are formed in the middle fluid as the inner fluid exits from the tapered end of the injection tube and contacts the middle fluid in the outer tube. As the inner and middle fluids continue to move into a collection tube disposed at an opposing end of the outer tube, where an outer fluid is introduced into the outer tube from the opposing end, smaller droplets of the inner fluid with a thin shell of the middle fluid are created, forming a double emulsion with the outer fluid flowing from the collection tube. In some embodiments, the tapered end of the injection tube is in close proximity to the inlet of the collection tube. FIG. 5B is a flow diagram showing formation of a water-in-oil-in-water double emulsion, using the process and/or device shown in FIG. 5A. The middle phase solvent is then evaporated to yield capsules. FIG. 5C is a flow diagram showing formation of a water-in-oil pre-emulsion, which is then introduced into the injection tube as shown in FIG. 5A as the inner fluid/phase. A double emulsion is formed using the process and/or device as shown in FIG. 5A. The shell (middle phase) and solvent of the inner phase can then be evaporated to yield capsules.

FIGS. 6A-6B are graphs showing injection force of microcapsules in an aqueous fluid versus a viscous solution. FIG. 6A shows injection force of a sample (comprising 80 μm PCL capsules dispersed in PBS at a volume fraction of 0.7) over time. FIG. 6B shows injection force of glycerol over time.

FIGS. 7A-7D show droplets generated according to one embodiment of the methods described herein. FIG. 7A shows droplets generated in the collection tube as shown in FIG. 5A. The encapsulation method is efficient and upscalable. The encapsulation efficiency is at least about 90% or higher, including 100%. In one embodiment, the encapsulation efficiency is 100%. The emulsification process is low shear. In some embodiments, the methods of making the droplets described herein can produce monodisperse emulsions and particles. FIG. 7B is a fluorescent image showing the shell of the droplets. FIG. 7C is a fluorescent image showing an active agent solution phase encapsulated in the droplets. FIG. 7D is a microscopic image showing the thickness of the shell, e.g., about 200 nm.

FIGS. 8A-8C are photographs showing upscalability of generation of double emulsion droplets at nozzles in a microfluidic Millipede single emulsion devices according to some embodiments described herein. FIG. 8A shows one embodiment of a Millipede single emulsion device. FIG. 8B shows another embodiment of a Millipede single emulsion device. FIG. 8C shows monodisperse droplets generated using those devices. In some embodiments, the drop-maker device can have about 500 nozzles. In some embodiments, the device can generate about 50 mL/hr dispersed phase. The devices described herein can be upscalable. Thus, the drop-maker device described herein can have more than 500 nozzles or can generate more than 50 mL/hr dispersed phase.

FIG. 9 is a set of time-lapse fluorescent images showing ability to have precise control release kinetics. The left column shows fast release of liquid active agent phase from emulsion droplets. The middle column shows shrinking of the emulsion droplet as the liquid active agent phase releases from the droplet. The right column shows that the droplet with a solid shell remain in size as the active agent releases from the droplet. The figure shows the ability of precise control over release kinetics in a time scale of seconds, minutes, or months.

FIGS. 10A-10C show biocompatibility of the encapsulation process. FIG. 10A shows encapsulation of 3T3 cells in gel droplets. FIG. 10B is a set of fluorescent images showing viability and growth of 3T3 cells in the gel droplets over a period of 14 days. FIG. 10C shows gel swelling upon exposure to different pHs.

FIGS. 11A-11C are photographs showing dropmaking according to some embodiments of the invention. FIG. 11A shows dropmaking at 80 cP with flow rates for the inner fluid, middle fluid and the outer fluid of 1,000 μl/hr, 1,000 μl/hr and 7,000 μl/hr respectively. FIG. 11B shows dropmaking at 200 cP with flow rates for the inner fluid, middle fluid and the outer fluid of 800 μl/hr, 1,000 μl/hr and 10,000 μl/hr respectively. FIG. 11C shows dropmaking at 600 cP with flow rates for the inner fluid, middle fluid and the outer fluid of 1,000 μl/hr, 1,000 μl/hr and 12,000 μl/hr respectively.

FIG. 12 is a graph showing the ratio of the flowrates of the inner fluid to middle fluid versus viscosity. Diamonds represent stable, single-core droplets in the data graph. At high ratios, the middle phase becomes too thin to maintain a stable droplet, and the droplet breaks (shown as the squares). At low ratios, multiple inner cores are injected into each individual droplet (shown as the triangles). The range to create stable, single core droplets appears to narrow slightly as the viscosity increases.

FIG. 13 is a graph showing the ratio of the flowrates of the inner fluid combined with the middle fluid to the outer fluid versus viscosity. Diamonds represent stable dropmaking, whereas, the squares represent regimes in which droplets are not made consistently. As viscosity increases, lower ratios appear to be necessary to make droplets. This demonstrates the necessity to increase the flow rate of the outer fluid in order to create enough force via flow focusing to break the stream into droplets.

### DETAILED DESCRIPTION OF THE INVENTION

Delivery of high concentration biologics or therapeutics has been a major challenge for pharmaceutical companies to expand their markets for biologics, e.g., protein-based therapeutics, such as monoclonal antibodies, to include homecare or self-injection. The challenge arises from, in part, the limited injection volume (e.g., no more than 5 mL or no more than 1.5 mL) available for subcutaneous administration, and the high viscosity associated with high concentration biologics or therapeutics to meet dosage requirements. Thus, there is a need for developing injectable compositions of biologics and/or therapeutic agents at high concentrations.

Embodiments of various aspects described herein relate to compositions, emulsions, devices, and methods for injecting a high viscosity and/or high concentration active agent solution. Examples of an active agent suitable to be administered using various aspects described herein include, but are not limited to, a protein, a peptide, an antibody, a growth factor, a nucleic acid, a sugar, an antigen, a vaccine, an enzyme, a cell, a small molecule covalently linked to a polymer, and a combination of two or more thereof. In particular, the compositions, emulsions, devices, and methods described herein are, in part, based on forming an emulsion in which liquid droplets comprising an active agent at a high concentration and/or a high viscosity are distributed in a lower viscosity injection solution. Thus, the required dose can be delivered subcutaneously through a small gauge needle (e.g., equal to or greater than 18 gauge, or about 25-30 gauge) in a typical injection volume (e.g., less than 5 mL or even less than 1.5 mL). In some embodiments, the droplets can comprise an outer shell or a solidified shell encapsulating a high viscosity of an active agent solution. These droplets can also confer additional stability to the encapsulated active agents such as drugs or bioactives. For example, the outer shell or solidified shell can protect the encapsulated active agent solution from external influences or environmental influences, including, e.g., light, change in pH, change in salinity or osmotic effects, and humidity, during administration, transport, handling, and/or storage. The outer shell or solidified shell can also be designed to allow for sustained release of the inner phase over a period of time, e.g., over hours, days, weeks or months. The outer shell or solidified shell can also be designed to allow for the administration into particular compartments of the body, e.g., the eye.

Low-Viscosity Compositions or Emulsions Comprising High-Concentration and/or High-Viscosity Active Agents

In one aspect, provided herein is a composition comprising: (i) droplets comprising a first liquid, the first liquid comprising an active agent at a concentration of at least about 50 mg/mL or higher; and (ii) a carrier liquid. The carrier liquid and the droplets are substantially immiscible, and the droplets are dispersed in the carrier liquid.

Generally, the viscosity of the first liquid comprising an active agent increases with the concentration of the active agent present in the first liquid. Thus, another aspect provided herein is a composition comprising: (i) droplets comprising a first liquid, the first liquid comprising an active agent and having a viscosity of at least about 20 cP or higher; and (ii) a carrier liquid. The carrier liquid and the droplets are substantially immiscible, and the droplets are dispersed in the carrier liquid.

As used herein throughout the specification, the term “liquid” or “liquid phase” refers to a substance or a composition in a physical state of matter that exhibits a characteristic of a flowable liquid at a temperature of about 4° C. to about room temperature and at an atmospheric pressure.

The term “viscosity” as used herein has its general meaning in the art. As used herein, the term “viscosity” refers to a measure of internal resistance of a liquid to a change in shape, or movement of neighboring portions relative to one another (a flow). It also corresponds to a common concept of “thickness” of a liquid. For example, honey has a much higher viscosity than water. When referring to the viscosity of a liquid comprising an active agent (e.g., a first liquid comprising an active agent), the term “viscosity” refers to apparent viscosity of the active-agent comprising liquid, not the inherent viscosity of the liquid without the active agent. The viscosity measurements are generally performed at room temperature or at a temperature of about 25° C., but they can also be performed at lower or higher temperatures. It is understood that the value of viscosity or apparent viscosity is dependent on the conditions under which the measurement was taken, such as temperature, the rate of shear and the shear stress employed. The apparent viscosity is defined as the ratio of the shear stress to the rate of shear applied under a specific condition. There are a number of alternative methods for measuring apparent viscosity or viscosity. For example, viscosity can be measured by a suitable cone and plate, parallel plate or other type of viscometer or rheometer.

In some embodiments of this aspect and other aspects described herein, the first liquid comprising an active agent (termed as “active agent-comprising first liquid” hereafter) can have a viscosity of about 0.8 cP to about 500 cP. In some embodiments, the first liquid comprising an active agent can have a viscosity of about 10 cP to about 500 cP, or about 20 cP to about 500 cP, or about 50 cP to about 500 cP. The viscosity measurements are generally measured at room temperature or at a temperature of about 25° C.

In some embodiments, the active agent-comprising first liquid can have a viscosity so high that it cannot be readily delivered by injection when it is injected alone (e.g., due to high concentrations of the active agent present in the first liquid or inherent viscosity of the active agent). For example, in some embodiments, the active agent-comprising first liquid can have a viscosity of at least about 50 cP or higher. In some embodiments, the active agent-comprising first liquid can have a viscosity of at least about 60 cP, at least about 70 cP, at least about 80 cP, at least about 90 cP, at least about 100 cP, at least about 200 cP, at least about 300 cP, at least about 400 cP, at least about 500 cP, or higher. In some embodiments, the active agent-comprising first liquid can have a viscosity of about 200 cP to about 500 cP. The viscosity measurements are generally measured at room temperature or at a temperature of about 25° C.

In accordance with various aspects described herein, when an active agent-comprising first liquid that is, by itself, injection-incompatible, is delivered in a form of droplets dispersed in an injection-suitable carrier liquid, the active agent can be delivered in liquid state at high concentrations and/or high viscosities, e.g., by injection, more readily, without pre-forming solid particles of the active agent.

Accordingly, it is desirable to select a carrier liquid with a viscosity low enough to facilitate delivery of viscous active agent-comprising droplets, e.g., by injection. In some embodiments, the carrier liquid can have a lower viscosity than that of the first liquid. In some embodiments, the viscosity of the carrier liquid can be selected such that it can be injected properly (e.g., using a syringe with a small needle). In some embodiments, the carrier liquid can have a viscosity of no more than 50 cP, no more than 45 cP, no more than 40 cP, no more than 35 cP, no more than 30 cP, no more than 25 cP, no more than 20 cP, no more than 15 cP, no more than 10 cP, no more than 5 cP, no more than 2 cP, no more than 1 cP. In some embodiments, the viscosity of the carrier liquid can have a range of about 0.8 cP to about less than 500 cP, e.g., when measured at room temperature or at a temperature of about 25° C. In some embodiments, the viscosity of the carrier liquid can have a range of about 0.8 cP to about 450 cP, e.g., when measured at room temperature or at a temperature of about 25° C. In some embodiments, the viscosity of the carrier liquid can have a range of about 0.8 cP to about 200 cP, e.g., when measured at room temperature or at a temperature of about 25° C.

As used herein, the term “carrier liquid” or “carrier liquid” refers to a liquid substance or composition in which droplets can be suspended or dispersed to yield an emulsion.

The compositions or emulsions described herein, as a whole (comprising droplets dispersed in a carrier liquid), can have a viscosity of no more than 100 cP, no more than 50 cP, no more than 45 cP, no more than 40 cP, no more than 35 cP, no more than 30 cP, no more than 25 cP, no more than 20 cP, or no more than 15 cP.

The carrier liquid and the droplets are substantially immiscible such that an emulsion is formed in which droplets are dispersed in the carrier liquid. Accordingly, in one aspect, described herein is also an emulsion comprising droplets dispersed in a carrier liquid, wherein the droplets comprise a first liquid, the first liquid comprising an active agent at a concentration of at least about 50 mg/mL; and the droplets and the carrier liquids are substantially immiscible. In another aspect, described herein is an emulsion comprising droplets dispersed in a carrier liquid, wherein the droplets comprise a first liquid, the first liquid comprising an active agent and having a viscosity of at least about 20 cP or at least about 50 cP; and the droplets and the carrier liquids are substantially immiscible.

As used herein, the term “emulsion” is a heterogeneous system comprising at least two or more substantially immiscible liquids, wherein one liquid is dispersed in another liquid in the form of droplets. By way of example only, emulsions can be biphasic systems comprising two immiscible liquid phases intimately mixed and dispersed with each other. Examples of an emulsion include, but are not limited to, water-in-oil emulsions, oil-in-water emulsion, water-in-water, water-in-oil-in-water emulsions, and oil-in-water-in-oil emulsions.

In some embodiments, an emulsion can be a microemulsion. As used herein, the term “microemulsion” refers to a thermodynamically stable, macroscopically homogeneous mixture of at least two substantially immiscible liquids and at least one surfactant. It contains, on a microscopic level, individual domains of two substantially immiscible phases separated by a surfactant layer. The decisive property is the thermodynamic stability. For a general description of microemulsions and their properties see “Surfactants and polymers in aqueous solution”, Jonsson B., Lindman B., Holmberg K., Kronberg B., Wiley&Sons Ltd, 1998, 365-399 (incorporated herein by reference).

In some embodiments, the emulsion is a stable emulsion. As used herein, the term “stable emulsion” refers to an emulsion in which droplets remain substantially evenly dispersed throughout a continuous phase (or a carrier liquid) for an extended time period (e.g., at least about 1 month or longer), including reasonable storage and usage times. For example, the droplets do not aggregate or settle out after an extended time period (e.g., at least about 1 month or longer).

As used herein, the term “substantially immiscible” refers to two or more liquids that do not form a homogenous mixture when they are in contact with each other. In some embodiments, when two or more substantially immiscible liquids are in contact with each other, one of the liquids can have a partial solubility (e.g., no more than 10% or lower) in another substantially immiscible liquid. The term “homogenous mixture” as used herein means that all components and/or liquids in a mixture are readily present in a single phase. For instance, one or more of the components and/or liquids do not separate into different phases even when the mixture is left stationary for an extended period of time (e.g., at least about 6 hours or longer, including, e.g., at least about 12 hours, at least about 18 hours, at least about 24 hours, or longer).

When referring to miscibility of the droplets and the carrier liquid, the term “substantially immiscible” refers to a liquid (e.g., a thin liquid layer) forming at least the outer surface of the droplets and the carrier liquid that do not form a homogenous mixture when they are in contact with each other.

As used herein and throughout the specification, the term “droplet” refers to a finite volume of matter comprising at least one liquid or at least one liquid phase, including, e.g., at least two or more liquids or liquid phases. The droplet comprises a high-concentration and/or a high-viscosity active agent in one or more liquid phases of the droplet. In some embodiments, when the droplet comprises a solid phase, the active agent is not present in the solid phase.

The droplets can be of any dimension, configuration, and/or shape provided that they are able to be delivered in the form of an emulsion to a target site, e.g., through a small orifice such as an injection needle. For example, in some embodiments of various aspects described herein, the droplets can have a droplet size that is smaller (e.g., at least 50% smaller) than the inner diameter of a needle that is used to deliver an emulsion comprising the droplets to a target site.

In some embodiments of various aspects described herein, the droplets can be microdroplets. As used herein, the term “microdroplets” refers to droplets having a droplet size of about 1 μm to about 1000 μm (e.g., in diameter). In some embodiments, the droplets can have a droplet size of about 1 μm to about 500 μm (e.g., in diameter). In some embodiments, the droplets can have a droplet size of about 10 μm to about 500 μm (e.g., in diameter). In some embodiments, the droplets can have a droplet size of about 10 μm to about 250 μm (e.g., in diameter). In some embodiments, the droplets can have a droplet size of about 10 μm to about 100 μm (e.g., in diameter). In some embodiments of various aspects described herein, the droplets can be nanodroplets. As used herein, the term “nanodroplets” refers to droplets having a droplet size of about 1 nm to about 1000 nm (e.g., in diameter).

It will be understood by one of ordinary skill in the art that droplets usually exhibit a distribution of droplet sizes around the indicated “size.” Unless otherwise stated, the term “droplet size” or “size” as used herein refers to the mode of a size distribution of droplets, i.e., the value that occurs most frequently in the size distribution. Methods for measuring the droplet size are known to a skilled artisan, e.g., by dynamic light scattering (such as photo-correlation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In some embodiments, the droplets in the compositions or emulsions described herein can have a narrow size distribution. The term “narrow size distribution” as used herein refers to a droplet size distribution that has a ratio of the volume diameter of the 90th percentile of droplets to the volume diameter of the 10th percentile less than or equal to 5. In some embodiments, the volume diameter of the 90th percentile of droplets to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1. In some embodiments, the droplets in the compositions or emulsions described herein can have substantially the same droplet size. In some embodiments, the droplets in the compositions or emulsions described herein can be monodisperse droplets. Methods for determining droplet size distribution are known in the art and can vary with the methods for measuring droplet size. In some embodiments, Geometric Standard Deviation (GSD) can be used to determine the droplet size distribution.

In some embodiments, the droplets in the compositions or emulsions described herein can have a broad size distribution. For example, the droplet size distribution can have a ratio of the volume diameter of the 90th percentile of droplets to the volume diameter of the 10th percentile greater than 5.

While the droplets can be of any shape, e.g., sphere, oval, elliptical, non-spherical, or irregular-shaped, in some embodiments of various aspects described herein, the droplets can have a substantially spheroidal morphology. In some embodiments, the particles can be substantially spherical. The term “substantially spherical” as used herein means that the ratio of the lengths of the longest to the shortest perpendicular axes of the droplet cross section is less than or equal to about 1.5. Substantially spherical does not require a line of symmetry. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in droplets that are substantially spherical, which minimizes the undesirable aggregation of the droplets upon storage. Unlike the droplets described herein, crystalline or non-crystalline solid particles can have more surfaces that can allow large surface contact areas where aggregation can occur, e.g., by ionic or non-ionic interactions. A sphere permits surface contact over a much smaller area.

In some embodiments, the droplets can be in a non-spherical or spherical shape resulting from fusion of at least two or more smaller droplets (e.g., 2, 3, 4, or more smaller droplets).

In some embodiments, the droplets can have a smooth surface.

In some embodiments, the droplets can have a rough surface. For example, in some embodiments where the droplets have a polymer shell, the surface roughness of the droplets can depend on the polymer.

The droplets in the compositions and/or emulsions described herein can have at least one or more liquid phases, including, e.g., at least two, or at least three liquid phases.

Single-phase droplets (single emulsion): In some embodiments, the droplets can be of a single liquid phase. In these embodiments, the droplets can be formed from a first liquid comprising at least one or more (e.g., at least two or more) active agent(s) as described herein. Thus, in some embodiments where the first liquid is an aqueous-based liquid, the single-phase droplets are aqueous-based droplets. In some embodiments, the corresponding carrier liquid can be an oil-based liquid. In some embodiments, the corresponding carrier liquid can be an aqueous-based liquid adapted to be incompatible to the droplets and the first liquid (e.g., an aqueous-based liquid comprising a water-soluble additive).

As used herein and throughout the specification, the term “aqueous-based liquid” refers to a liquid comprising at least water, e.g., at least about 50% (w/w) water or more. In some embodiments, the aqueous-based liquid can have at least about 60% (w/w) water or more, including, e.g., at least about 70% (w/w) water, at least about 80% (w/w) water, at least about 90% (w/w) water, at least about 95% (w/w) water, or more. In some embodiments, an aqueous-based liquid, e.g., as part of a liquid phase within the droplets and/or the carrier liquid can include, but is not limited to, a buffered solution. In some embodiments, the aqueous-based liquid can further comprise a water-soluble additive. In one embodiment, the water-soluble additive can comprise a water-soluble polymer. Non-limiting examples of a water-soluble polymer can include, but are not limited to, cellulose, dextran, poly acrylic acid (PAA), poly(ethylene glycol) (PEG), poly(vinyl acetate), polyvinyl alcohol (PVA), poly(lactic acid)(PLA), polyhydroxy ethyl methacrylate, polyacrylamide, polyethylene oxide, alginate, Polyacrylamide, poly(acrylic acid), poly(methacrylic acid), poly(2-ethyl-2-oxazoline), polyethylenimine (PEI), Pluronic F68, Triton X-100, Tween 20, poly(propylene glycol), N,N-dimethyldodecylamine N-oxide, Zonyl, Ficoll, dextran, poly(2-ethyl-2-oxazoline), hydroxyethyl cellulose, Brij 35, methyl cellulose, polyvinylpyrrolidone (PVP), Zonyl, poly(acrylic acid), alginic acid, sodium dodecyl sulfate-polymer (SDS), diethylaminoethyl-dextran, poly(methacrylic acid), carboxy-polyacrylamide, dextran sulfate, carboxy-polyacrylamide, and a combination of two or more thereof.

In some embodiments where the first liquid is a non-aqueous-based liquid (e.g., an oil-based liquid), the single-phase droplets are non-aqueous-based droplets (e.g., oil-based droplets). In some embodiments, the corresponding carrier liquid can be an aqueous-based liquid. In some embodiments, the corresponding carrier liquid can be a non-aqueous-based liquid adapted to be incompatible to the droplets and the first liquid.

As used herein and throughout the specification, the term “non-aqueous-based liquid” refers to a liquid comprising at least 50% (w/w) or more organic liquid or solvent. In some embodiments, the non-aqueous-based liquid can have at least about 60% (w/w) organic liquid or solvent or more, including, e.g., at least about 70% (w/w) organic liquid or solvent, at least about 80% (w/w) organic liquid or solvent, at least about 90% (w/w) organic liquid or solvent, at least about 95% (w/w) organic liquid or solvent, or more. Examples of an organic liquid or solvent include, but are not limited to, a liquid lipid or fatty acid ester or alcohol (propylene glycol dicaprylate/dicaprate), oil, or other organic compound such as benzyl benzoate or ethyl lactate. In some embodiments, the non-aqueous-based liquid can comprise an additive as described herein.

In one embodiment of various aspects described herein, the non-aqueous-based liquid can be an oil-based liquid. The term “oil” as used herein refers to any glyceride of a fatty acid (e.g., mono-, di-, or triglycerides) that is capable of being converted into esters of the fatty acid by a transesterification reaction. The term “oil” as used herein refers to a liquid state material at room temperature. The term “oil” as used herein can include oils from any of animal, plant or synthetically-derived sources. In some embodiments, an oil-based liquid, e.g., as part of a liquid phase within the droplets and/or the carrier liquid can include, but is not limited to, ethyl acetate, omega-3 oil, fish oil, silicone oil, parenteral oil (e.g., but not limited to LIPOSYN parenteral oil), mineral oil, paraffin, fatty esters, olive oil, dichloromethane, sunflower oil, fluorinated oils, perfluoro alkanes, and a combination of two or more thereof.

In some embodiments, the oil-based liquid can further comprise an oil-soluble additive. In one embodiment, the oil-soluble additive can comprise an oil-soluble polymer. Exemplary oil-soluble polymers can include, without limitations, the oil-soluble polymer comprises polycaprolactone (PCL), poly (N-vinyl pyrolidone), poly glycolic acid (PGA), poly lactic-co-glycolic acid (PLGA), poly L-lactic acid (PLLA), poly propylene fumarate (PPF), polybutadiene, polyisoprene, and a combination of two or more thereof.

The oil can be volatile or involatile. In some embodiments, the oil can be volatile, e.g., oil that is able to be evaporated or removed upon generation of droplets as described herein. In these embodiments, when oil comprising a polymer (e.g., biodegradable polymer such as poly(lactic acid)) forms the shell of a droplet, the oil can be evaporated or removed to form droplets comprising a liquid core with a high-concentration active agent solution, and a polymeric shell surrounding the liquid core.

Droplets with two or more liquid phases (double or multiple higher-order emulsion): In some embodiments, the droplets can each have at least two immiscible liquid phases or more. The two or more liquid phases within the droplets can be arranged in various configurations known in the art. By way of example only, in some embodiments, the droplets can each comprise a first liquid and a second liquid, wherein the first liquid and the second liquid are substantially immiscible. In some embodiments, the first liquid can form the core of the droplets while the second liquid can form a shell surrounding the core. In some embodiments, the second liquid can instead form the core of the droplets while the first liquid can form a shell surrounding the core). Other configurations of multiple phases within the droplets are also applicable. For example, in some embodiments, the droplets are formed from a second liquid, in which the first liquid in the form of subdroplets are dispersed. Alternatively, the droplets can comprise a first liquid, in which the second liquid in the form of subdroplets are dispersed.

Droplets of different combinations of immiscible liquid phases (e.g., the first and second liquids and carrier liquid) can be formed provided that any two liquid phases that are selected to be in contact with each other form an interface. In some embodiments, the first liquid can be an aqueous-based liquid as described herein. The corresponding second liquid can be a non-aqueous-based liquid as described herein (e.g., an oil-based liquid), or alternatively, an aqueous-based liquid adapted to be incompatible with the first liquid. Depending on the arrangements of the first liquid and the second liquid within the droplets, e.g., the chemical nature of the liquid phase on the outer surface of the droplets, the carrier liquid can be an aqueous-based liquid or a non-aqueous liquid (e.g., an oil-based liquid). By way of example only, when the outer surface of the droplets is a non-aqueous-based liquid (e.g., an oil-based liquid), the carrier liquid can be an aqueous-based liquid, or alternatively, a non-aqueous-based liquid adapted to be incompatible with the liquid on the outer surface of the droplets.

As used herein and throughout the specification, the term “adapted to be incompatible” or “incompatible” when referring to a liquid (e.g., a first liquid, a second liquid, and/or a carrier liquid) means that the liquid can be formulated to comprise an additive as described herein, e.g., a polymer, to create an interfacial tension between the liquid and another liquid when they are in contact with each other such that an emulsion is formed. By way of example only, when two liquid of the same nature (e.g., both aqueous-based or both non-aqueous-based) are in contact with each other, an interfacial tension between the two liquid phases can be created by altering the surface tension of at least one or both of the two liquids. For example, at least one or both of the two liquids can be formulated to comprise a soluble polymer to alter its surface tension such that an interfacial tension is created to cause formation of an emulsion. In some embodiments, the first liquid or the second liquid can be an aqueous-based liquid comprising a first water-soluble polymer, and the carrier liquid can be an aqueous-based liquid comprising a second water-soluble polymer. The first water-soluble polymer and the second water-soluble polymer can be selected to be incompatible to each other such that an interfacial tension is created between the resulting liquid phases to cause formation of an emulsion. For example, the first water-soluble polymer and the second water-soluble polymer can each be independently selected from the group consisting of cellulose, dextran, poly acrylic acid (PAA), poly(ethylene glycol) (PEG), poly(vinyl acetate), polyvinyl alcohol (PVA), poly(lactic acid)(PLA), polyhydroxy ethyl methacrylate, polyacrylamide, polyethylene oxide, alginate, Polyacrylamide, poly(acrylic acid), poly(methacrylic acid), poly(2-ethyl-2-oxazoline), polyethylenimine (PEI), Pluronic F68, Triton X-100, Tween 20, poly(propylene glycol), N,N-dimethyldodecylamine N-oxide, Zonyl, Ficoll, dextran, poly(2-ethyl-2-oxazoline), hydroxyethyl cellulose, Brij 35, methyl cellulose, polyvinylpyrrolidone (PVP), Zonyl, poly(acrylic acid), alginic acid, sodium dodecyl sulfate-polymer (SDS), diethylaminoethyl-dextran, poly(methacrylic acid), carboxy-polyacrylamide, dextran sulfate, carboxy-polyacrylamide, and a combination of two or more thereof.

Examples of different combinations of the first water-soluble polymer and the second water-soluble polymer that can be used in the first liquid and carrier liquid, respectively, to generate an emulsion include, but are not limited to, polyacrylamide & poly(acrylic acid); polyacrylamide & poly(methacrylic acid); polyacrylamide & poly(ethylene glycol) (PEG); polyacrylamide & poly(2-ethyl-2-oxazoline); polyacrylamide & polyethylenimine (PEI); polyacrylamide & polyvinyl alcohol (PVA); polyacrylamide & Pluronic F68; polyacrylamide & Triton X-100; polyacrylamide & Tween 20; polyacrylamide & poly(propylene glycol); polyacrylamide & N;N-dimethyldodecylamine N-oxide; polyacrylamide & Zonyl; Ficoll & poly(methacrylic acid); Ficoll & dextran; Ficoll & PEG; Ficoll & poly(2-ethyl-2-oxazoline); Ficoll & PEI; Ficoll & PVA; Ficoll & hydroxyethyl cellulose; Ficoll & Pluronic F68; Ficoll & Triton X-100; Ficoll & Tween 20; Ficoll & Brij 35; Ficoll & methyl cellulose; dextran & Ficoll; dextran & poly(2-ethyl-2-oxazoline); dextran & PEG; dextran & PVA; hydroxyethyl cellulose; dextran & Pluronic F68; dextran & Triton X-100; dextran & Tween 20; dextran & poly(propylene glycol); dextran & polyvinylpyrrolidone (PVP); dextran & Zonyl; poly(acrylic acid) & polyacrylamide; poly(acrylic acid) & PEG; poly(acrylic acid) & alginic acid; poly(acrylic acid) & sodium dodecyl sulfate-polymer (SDS); poly(acrylic acid) & diethylaminoethyl-dextran; poly(methacrylic acid) & polyacrylamide; poly(methacrylic acid) & Ficoll; poly(methacrylic acid) & PEG; poly(methacrylic acid) & poly(2-ethyl-2-oxazoline); poly(methacrylic acid) & PEI; poly(methacrylic acid) & Pluronic F68; poly(methacrylic acid) & Triton X-100; poly(methacrylic acid) & Tween 20; poly(methacrylic acid) & carboxy-polyacrylamide; poly(methacrylic acid) & poly(propylene glycol); poly(methacrylic acid) & PVP; poly(methacrylic acid) & N;N-dimethyldodecylamine N-oxide; poly(methacrylic acid) & Zonyl; PEG & polyacrylamide; PEG & Ficoll; PEG & dextran; PEG & poly(acrylic acid); PEG & poly(methacrylic acid); PEG & PEI; PEG & PVA; PEG & poly(2-ethyl-2-oxazoline); PEG & Tween 20; PEG & dextran sulfate; PEG & PVP; PEI & polyacrylamide; PEI & Ficoll; PEI & poly(methacryilc acid); PEI & PEG; PEI & poly(2-ethyl-2-oxazoline); PEI & Pluronic F68; PEI & carboxy-polyacrylamide; PVA & polyacrylamide; PVA & Ficoll; PVA & dextran; PVA & PEG; PVA & PEI; PVA & Pluronic F68; PVA & Tween 20; PVA & dextran sulfate; PVA & carboxy-polyacrylamide; hydroxyethyl cellulose & Ficoll; hydroxyethyl cellulose & dextran; hydroxyethyl cellulose & Triton X-100; and hydroxyethyl cellulose & Tween 20.

In some embodiments, double emulsions can be used to prepare droplet structures with an aqueous core (e.g., a water core), a biocompatible non-aqueous shell (e.g., a biocompatible oil shell), and an aqueous continuous phase (e.g., a water carrier liquid). The core can comprise a high-concentration and/or high-viscosity active agent solution with any optional additives described herein, e.g., added to ensure stability. The non-aqueous shell can comprise oil or a polymer. The polymer can be selected not only to ensure encapsulation of active agent(s), but also to control release of the active agent(s) to enable extended or delayed release, if desired. In some embodiments, the release kinetics can be tuned to a time scale of seconds, minutes, or months.

In alternative embodiments, the shell can comprise a solvent that can be subsequently evaporated to form either a liposome or a polymersome structure to make highly biocompatible encapsulation structures in a water-continuous phase. Examples of biocompatible solvents that can be evaporated include, but are not limited to, dichloromethane, ethyl acetate, or a combination thereof.

Polymersomes can comprise block-copolymers, dendritic polymers, or brush polymers that have hydrophobic and hydrophilic segments. Non-limiting examples of hydrophilic segments include poly(ethylene glycol), poly(2-methyloxazoline), poly(acrylic acid), and a combination of two or more thereof. Non-limiting examples of hydrophobic segments include polydimethylsiloxane, poly(caprolactone), poly(lactide), poly(methyl methacrylate), and a combination of two or more thereof.

Liposomes and polymersomes can be unilamellar or multilamellar. Examples of compounds/lipids for vesicles include, but are not limited to, phospholipids, ammonium salts such as dimethyldioctadecylammonium chloride, benzethonium chloride, polyethoxylated tallow amine, cetylpyridinium chloride, cetrimonium bromide, dioctadecyldimethylammonium bromide, or a combination of two or more thereof.

Droplets in a form of microcapsules: In some embodiments, the droplets with two or more liquid phases as described herein (e.g., double emulsion) can be further processed to obtain capsules or microcapsules. For example, in some embodiments, the middle phase of a double emulsion can be solidified to obtain capsules or microcapsules. Accordingly, in some embodiments, the droplets can each comprise a first phase and a second phase, where the first phase and the second phase are substantially immiscible, and the first phase forms the core of the droplets while the second phase forms a shell surrounding the core. The size of the droplets and/or core-shell ratio can vary and be precisely controlled to suit the need of an application. In some embodiments, the size of the droplets can range from about 5 μm to about 500 μm, or about 10 μm to about 300 μm, or about 20 μm to about 200 μm. In some embodiments, the shell of the droplets can have a thickness of about 50 nm to about 700 nm, or about 100 nm to about 500 nm, or about 150 nm to about 250 nm. In one embodiment, the shell of the droplets can have a thickness of about 200 nm. In some embodiments, the core-shell ratio can range from about 50:1 to about 2000:1, or about 100:1 to about 1000:1.

Solidification of the middle phase or the second phase can occur through any methods known in the art, including, e.g., but not limited to solvent evaporation, non-covalent cross-linking, covalent cross-linking, interfacial polymerization, electrostatic interactions, coacervation, or a combination of two or more thereof.

The solidified middle phase or second phase or shell can not only be used to encapsulate or hide a high viscosity of an active agent solution, but can also confer additional stability to the encapsulated active agents such as drugs or bioactives. For example, the solidified shell can protect the encapsulated active agent solution from external influences or environmental influences, including, e.g., light, change in pH, change in salinity or osmotic effects, and/or humidity, during administration, transport, handling, and/or storage.

In some embodiments, the capsules or microcapsules can enable controlled or sustained release of at least one or more active agent(s) encapsulated therein.

The resulting capsules or microcapsules can then be dispersed in a suitable carrier fluid before injection. In some embodiments, such dispersions can contain a volume fraction of capsules or microcapsules up to 0.9 or lower, including, e.g., 0.85, 0.8, 0.75, 0.7, or lower. In one embodiment, the dispersion of capsules or microcapsules in a carrier fluid (e.g., a low viscosity carrier fluid) can contain a volume fraction of capsules or microcapsules up to about 0.74.

To fabricate capsules or microcapsules, the middle phase of the double emulsion template or the second phase of the droplets generally contains at least one or more (e.g., at least two, at least three or more) polymers or polymer precursors. Non-limiting examples of polymers that can be included into the middle phase or the second phase to form microcapsules can include polylactic acid (PLA), poly(lactic-co-glycolic acid) (PLGA), polycaprolactone, cellulose, chitosan, gelatin, guar, shellac, or a combination of two or more thereof. Other appropriate polymers described herein can also be included into the middle phase or the second phase to form microcapsules.

Encapsulation of preformed emulsions: In some embodiments, capsules or microcapsules can be formed using a preformed emulsion as an inner phase of the double emulsion, at least one or more (including, e.g., at least two, at least three or more) shell-forming polymers dissolved in an appropriate solvent as middle phase, and a suitable surfactant dissolved in an aqueous solvent (e.g., water) as an outer continuous phase. Accordingly, in some embodiments, capsules or microcapsules can be formed using a preformed emulsion as the first phase of the droplets, at least one or more (including, e.g., at least two, at least three or more) shell-forming polymers or polymer precursors described herein dissolved in an appropriate solvent as the second phase, and a suitable surfactant dissolved in an aqueous solvent (e.g., water) as an outer continuous phase or carrier liquid.

In some embodiments, the inner phase of a double emulsion or the first phase of the droplets can comprise, consist essentially of, or consist of a viscous aqueous phase dispersed in an evaporable or volatile organic phase. In some embodiments, the evaporable or volatile organic phase can further comprise a surfactant. Examples of the evaporable or volatile organic phase include, but are not limited to perfluorohexane, dichloromethane, ethanol, ethyl acetate, or a combination of two or more thereof. The aqueous phase comprises at least one or more (including, e.g., at least two or more) (bio)active encapsulants or active agents, wherein at least one of which is present at a high concentration. The concentration(s) of the (bio)active encapsulant(s) or active agent(s) can be so high that the solution would be too viscous to be administered as a viscous liquid by itself.

The ratio of the viscous aqueous phase to the evaporable or volatile organic solvent in the preformed emulsion can vary dependent on a desired application. In some embodiments, the volume ratio of an aqueous phase to an evaporable or volatile organic solvent in a pre-emulsion can range from about 1:10 to about 10:1. In some embodiments, the volume ratio of an aqueous phase to an evaporable or volatile organic solvent in a pre-emulsion can be about 1:0.3, 1:0.4, 1:0.5, 1:0.6, 1:0.7, 1:0.8, 1:0.9, 1:1, 1:2, 1:3, 1:4, 2:3, 2:4, or 3:4.

The preformed emulsion or pre-emulsion can be formed by any methods known in the art, including, e.g., but not limited to shaking, vortex emulsification, ultrasound emulsification, spontaneous emulsification, membrane emulsification, vibrating nozzle emulsification, high pressure homogenization, mechanical homogenization, rotor stator homogenization, magnetic stirring, mechanical stirring, static mixing, using a microfluidic device, or a combination of two or more thereof.

Encapsulation of preformed dispersions: In some embodiments, capsules or microcapsules can be formed using a preformed dispersion as an inner phase of the double emulsion, at least one or more (including, e.g., at least two, at least three or more) shell-forming polymers dissolved in an appropriate solvent as a middle phase, and a suitable surfactant dissolved in an aqueous solvent (e.g., water) as an outer continuous phase. Accordingly, in some embodiments, capsules or microcapsules can be formed using a preformed dispersion as the first phase of the droplets, at least one or more (including, e.g., at least two, at least three or more) shell-forming polymers or polymer precursors described herein dissolved in an appropriate solvent as the second phase, and a suitable surfactant dissolved in an aqueous solvent (e.g., water) as an outer continuous phase or carrier liquid.

In some embodiments, the inner phase of a double emulsion or the first phase of the droplets can comprise, consist essentially of, or consist of solid particles dispersed in an organic phase or aqueous phase. In some embodiments, the organic phase can be evaporable or volatile. Examples of the organic phase include, but are not limited to perfluorohexane, dichloromethane, ethanol, ethyl acetate, or a combination of two or more thereof. In some embodiments, the organic phase can further comprise one or more surfactants, one or more stabilizing polymers, stabilizing colloidal particles, or a combination of two or more thereof. Non-limiting examples of stabilizing polymers include, but are not limited to polyethylene glycol, polyvinylpyrrolidone (PVP), polyethylene glycol-b-polypropylene glycol-b-polyethylene glycol, polypropylene glycol-b-polyethylene glycol-b-polypropylene glycol. In one embodiment, the stabilizing colloidal particles can comprise silica particles. In some embodiments, the aqueous phase used to disperse the solid particles can comprise water and/or a buffered solution.

The solid particles can comprise, consist essentially of, or consist of at least one or more pure active agents, drugs, or bioactives. Alternatively or additionally, the solid particles can comprise, consist essentially of, or consist of at least one or more active agents, drugs, or bioactives, wherein at least one of the active agents, drugs, or bioactives is entrapped in a matrix at a high concentration. The concentration(s) of the active agent(s), drug(s), or bioactive(s) entrapped in a matrix can be so high that the matrix mixture would be too viscous to be administered by itself. Examples of a matrix entrapping an active agent, drug, or bioactive include, but are not limited to gelatin, alginate, chitosan, guar, PLGA, PLA, polycaprolactone, or a combination of two or more thereof. Methods to fabricate such particles comprising an active agent, drug, or bioactive are known in the art, including, e.g., coacervation, spray drying, solvent evaporation, precipitation, extrusion, and a combination of two or more thereof. The size of the dispersed active agent-comprising solid particles can vary with desired applications and ranges from nanometers to micrometers. In some embodiments, the size of the dispersed active agent-comprising solid particles can range from about 10 nm to about 10 μm, or from about 20 nm to about 5 μm.

The ratio of the solid particles to the organic phase or aqueous phase in the preformed dispersion can vary dependent on a desired application. In some embodiments, the volume fraction of solid particles in a pre-formed dispersion can range from about 0.1 to about 0.74.

In various embodiments of the aspects described herein, the droplets and the carrier liquid can constitute the compositions described herein in any volume ratio provided that there is sufficient volume of the carrier liquid, or the composition as a whole has an apparent viscosity that is low enough, to facilitate delivery of the droplets to a target site, e.g., through a small orifice such as an injection needle. Generally, the higher the volume ratio of the droplets to the carrier liquid, the higher amount of an active agent can be present in the compositions. In some embodiments, the droplets and the carrier liquid can be in a volume ratio of about 10:90 to about 70:30. In some embodiments, the droplets and the carrier liquid can be in a volume ratio of about 10:90, about 20:80, about 30:70, about 40:60, about 50:50, about 60:40, or about 70:30.

In some embodiments of this aspect and other aspects described herein, the concentration of the active agent(s) within the droplets, e.g., present in the first liquid and/or in the second liquid can be at least about 75 mg/mL or higher. In some embodiments, the concentration of the active agent(s) within the droplets, e.g., present in the first liquid and/or in the second liquid can be at least about 100 mg/mL, at least about 110 mg/mL, at least about 120 mg/mL, at least about 130 mg/mL, at least about 140 mg/mL, at least about 150 mg/mL, at least about 160 mg/mL, at least about 170 mg/mL, at least about 180 mg/mL, at least about 190 mg/mL, at least about 200 mg/mL, at least about 250 mg/mL, at least about 300 mg/mL, at least about 350 mg/mL, at least about 400 mg/mL, at least about 450 mg/mL, at least about 500 mg/mL, at least about 600 mg/mL, at least about 700 mg/mL, at least about 800 mg/mL, at least about 900mL, or higher.

In some embodiments, the composition, the first liquid, and/or the carrier liquid can further comprise one or more (e.g., two or more) additives. An additive can be selected, for example, to reduce or minimize aggregation and/or denaturation of an active agent, to stabilize the dispersion of droplets in a carrier liquid, to resist pH changes, and/or to adjust osmolality. Accordingly, an additive can include, but is not limited to, a stabilizer, an emulsifier, a surfactant, a sugar, a polyol (for example, glycerol, propylene glycol, liquid polyethylene glycol, and the like), an amino acid, a buffered solution, a chelating agent, and/or a polymer. Non-limiting examples of a surfactant include polyvinylalcohol, Tween 80, sodium dodecyl sulfate, or a combination of two or more thereof. Polymer(s) that can be added as additive(s) into the composition, the first liquid, and/or the carrier liquid can be any water-soluble polymer described herein or oil-soluble polymer described herein. Examples of a water-soluble polymer to be added as an additive include, but are not limited to, PEG-based polymer, dextran, or a combination of both. In some embodiment, the polymer(s) that can be added as additive(s) into the composition, the first liquid, and/or the carrier liquid can include hydrogel. Exemplary hydrogels include, but are not limited to, alginate, gelatin, guar, PEG-based hydrogels, or a combination of two or more thereof.

Additional examples of additives that can be added to the composition, the first liquid, and/or the carrier liquid include, without limitations, salts, sugars, organics, buffers, polymers and other compositions that include: Disodium edetate, Sodium chloride, Sodium citrate, Sodium succinate, Sodium hydroxide, Sodium glucoheptonate, Sodium acetyltryptophanate, Sodium bicarbonate, Sodium caprylate, Sodium pertechnetate, sodium acetate, sodium dodecyl sulfate, aluminum hydroxide, aluminum phosphate, ammonium citrate, calcium chloride, calcium, potassium chloride, potassium sodium tartarate, zinc oxide, zinc, stannous chloride, magnesium sulfate, magnesium stearate, titanium dioxide, DL-lactic/glycolic acids, asparagine, L-arginine, arginine hydrochloride, adenine, histidine, glycine, glutamine, glutathione, imidazole, protamine, protamine sulfate, phosphoric acid, Tri-n-butyl phosphate, ascorbic acid, cysteine hydrochloride, hydrochloric acid, hydrogen citrate, trisodium citrate, guanidine hydrochloride, mannitol, lactose, sucrose, agarose, sorbitol, maltose, trehalose, surfactants, polysorbate 80, polysorbate 20, poloxamer 188, sorbitan monooleate, triton n101, m-cresol, benzyl alcohol, ethanolamine, glycerin, phosphorylethanolamine, tromethamine, 2-phenyloxyethanol, chlorobutanol, dimethylsulfoxide, N-methyl-2-pyrrolidone, propyleneglycol, Polyoxyl 35 castor oil, methyl hydroxybenzoate, tromethamine, corn oil-mono-di-triglycerides, poloxyl 40 hydrogenated castor oil, tocopherol, n-acetyltryptophan, octa-fluoropropane, castor oil, polyoxyethylated oleic glycerides, polyoxytethylated castor oil, phenol (antiseptic), glyclyglycine, thimerosal (antiseptic, antifungal), Parabens (preservative), Gelatin, Formaldehyde, Dulbecco's modified eagles medium, Hydrocortisone, Neomycin, Von Willebrand factor, Gluteraldehyde, Benzethonium chloride, White petroleum, p-aminopheyl-p-anisate, monosodium glutamate, beta-propiolactone, Acetate, Citrate, Glutamate, Glycinate, Histidine, Lactate, Maleate, Phosphate, Succinate, Tartrate, Tris, Carbomer 1342 (copolymer of acrylic acid and a long chain alkyl methacrylate cross-linked with allyl ethers of pentaerythritol), Glucose star polymer, Silicone polymer, Polydimethylsiloxane, Polyethylene glycol, carboxymethylcellulose, Poly(glycolic acid), Poly(lactic-co-glycolic acid), Polylactic acid, Dextran 40, Poloxamers (triblock copolymers of ethylene oxide and propylene oxide), and a combination of two or more thereof.

In some embodiments, the additive(s) can be present in the composition, the first liquid, and/or the carrier liquid at a concentration of about 0.0001% to about 5.0%, or about 0.001% to about 2.5%, or about 0.01% to about 1%, or about 0.01% to about 0.1%.

In some embodiments, the composition, the first liquid, and/or the carrier liquid can comprise at least one or more stabilizers. A “stabilizer” herein is an excipient, or mixture of two or more excipients, which stabilizes the compositions and/or active agent(s) present in the droplets. For example, the stabilizer can prevent instability of the compositions or active agent(s) described herein during droplet and/or emulsion fabrication methods. Exemplary stabilizers include, but are not limited to, saccharides, surfactants, amino acids, and a combination of two or more thereof.

A “saccharide” as defined herein comprises the general composition (CH₂O)ₙ and derivatives thereof, including monosaccharides, disaccharides, trisaccharides, polysaccharides, sugar alcohols, reducing sugars, nonreducing sugars, etc. Examples of saccharides include glucose, sucrose, trehalose, lactose, fructose, maltose, dextran, glycerin, dextran, erythritol, glycerol, arabitol, sylitol, sorbitol, mannitol, mellibiose, melezitose, raffmose, mannotriose, stachyose, maltose, lactulose, maltulose, glucitol, maltitol, lactitol, iso-maltulose, and a combination of two or more thereof.

In some embodiments, the composition, the first liquid, and/or the carrier liquid can comprise at least one or more emulsifiers. As used herein, the term “emulsifier” refers to a surface active component comprising one or more substances having a polar or ionic portion and a non-polar, e.g., aliphatic portion, which surface active component is capable of stabilizing an emulsion. Examples of emulsifiers include, but are not limited to egg yolk, lecithin, sodium stearoyl lactylate, diacetyl tartaric (acid) ester of monoglyceride, acetyl alcohol, polysorbate 20, ceteareth 20, vitamin E and derivatives thereof, and a combination of two or more thereof.

In some embodiments, the composition, the first liquid, and/or the carrier liquid can comprise at least one or more surfactants. As used herein, the term “surfactant” refers to an agent to lower the surface tension between two liquids, e.g., a nonionic surfactant. Non-limiting examples of surfactants include polysorbate (for example, polysorbate 20 and, polysorbate 80); poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g. Pluronics, PF68 etc); and a combination of two or more thereof. The surfactant may be included to prevent or reduce aggregation or denaturation of the monoclonal antibody in the preparation and/or formulation.

In some embodiments of this aspect and other aspects described herein, the composition does not include a viscosity-reducing agent, e.g., an agent to reduce viscosity. Exemplary viscosity-reducing agent is proline.

In some embodiments of this aspect and other aspects described herein, the compositions or emulsions described herein are formulated to be “injectable”. The “injectability” of the compositions or emulsions described herein refers to the ease with which the compositions or emulsions can be administered to a subject. In various embodiments, the injectability of a given composition or emulsion described herein can be superior to the injectability of an equivalent liquid formulation comprising the same active agent concentration and the same excipient(s) and concentration(s) thereof.

In one embodiment, the injectability of the compositions or emulsions described herein can be evaluated based on duration of time to inject a certain volume of a single dose (e.g., a volume of no more than 5 mL or less, including, e.g., no more than 4 mL, no more than 3 mL, no more than 2 mL, no more than 1.5 mL, no more than 1 mL or lower) to a target site. In some embodiments, injection of a single dose of the compositions or emulsions (e.g., having a volume of no more than 5 mL or less, including, e.g., no more than 4 mL, no more than 3 mL, no more than 2 mL, no more than 1.5 mL, no more than 1 mL or lower) to a target site (e.g., by subcutaneous injection) can be performed in less than 5 minutes or shorter, including, e.g., less than 4 minutes, less than 3 minutes, less than 2 minutes, less than 1 minute, less than 30 seconds, less than 20 seconds, less than 10 seconds, less than 5 seconds or shorter. In some embodiments, injection of a single dose of the compositions or emulsions (e.g., having a volume of no more than 5 mL or less, including, e.g., no more than 4 mL, no more than 3 mL, no more than 2 mL, no more than 1.5 mL, no more than 1 mL or lower) to a target site (e.g., by subcutaneous injection) can be performed in less than 1 minute or shorter, including, e.g., less than 45 seconds, less than 30 seconds, less than 20 seconds, less than 10 seconds, less than 5 seconds or shorter.

In one embodiment, the injectability of the compositions or emulsions described herein can be evaluated based on the injection glide force. The term “injection glide force” as used herein refers to the force required for the injection of a solution at a given injection rate via a needle of predetermined gauge and length. In one embodiment, it is evaluated using a pre-filled syringe (e.g., 1.0 mL-volume syringe with a needle having a gauge of at least 18 or higher, or about 25-30) with glide force analyzed and established as a function of the distance of the plunger rod travelling inside the syringe at a steady compression rate. Time and force required for a manual injection (or time required for an injection using an autoinjector) may impact the usability of the product by the end-user (and thus compliance with the intended use of the product). In one embodiment, the Hagen-Poiseuille equation can be utilized to estimate the travel (or glide) force (Equation 1).

$$F = \frac{8 Q μ L}{π {R}^{4}} \times A \qquad ( Equation 1 )$$

where Q=Volumetric flow rate

μ=Fluid viscosity

L=Needle length

R=Needle inner diameter

A=Cross sectional area of syringe plunger

F=Frictionless travel force

According to Equation 1, the glide force is dependent on a number of parameters. Compositions or emulsions with a high viscosity can lead to high injection forces and long injection times since both parameters are proportional to viscosity. Generally accepted limits for injection force and injection time can depend e.g., on the indication and the dexterity of the patient population. In some embodiments, the injection glide force of the compositions or emulsions described herein can be about 20 newtons or less. In some embodiments, the injection glide force of the compositions or emulsions described herein can be about 15 newtons or less. In some embodiments, the injection glide force can be from about 2 newtons to about 20 newtons. In some embodiments, the injection glide force can be from about 2 newtons to about 15 newtons.

In some embodiments, the injection pressure of the compositions or emulsions described herein can be no more than 200 mPa or lower. In some embodiments, the injection pressure of the compositions or emulsions described herein can be no more than 175 mPa or lower, including, e.g., no more than 160 mPa, no more than 150 mPa, no more than 125 mPa, no more than 100 mPa, no more than 75 mPa or lower. Methods for evaluating injectability of parenteral dosage form are known in the art. See, e.g., Cilurzo et al., AAPS PharmSciTech (2011) 12(2): 604-609.

The compositions and emulsions described herein are generally sterile, and this can be achieved according to the procedures known to a skilled person for generating sterile pharmaceutical formulations suitable for administration to human subjects, including, e.g., but no limited to, filtration through sterile filtration membranes, prior to, or following, preparation of the compositions and emulsions described herein. Moreover, the compositions and emulsions described herein are desirably ones which have been demonstrated to be stable upon storage. Various stability assays are available to the skilled practitioner for confirming the stability of the compositions and emulsions described herein. Stability can be tested by evaluating physical stability, chemical stability, and/or biological activity of active agent(s) present in the compositions and emulsions described herein around the time of formulation as well as following storage at different temperatures and time-points.

Methods for Producing Injectable Composition Comprising High-Concentration and/or High-Viscosity Active Agents

Injection of a high-concentration therapeutics (e.g., at a concentration of 50 mg/mL or higher, including, e.g., at a concentration of 100 mg/mL or higher) can interfere with the plunging action of syringes, as the high-concentration therapeutics generally tend to be viscous. To overcome such problem, high-concentration and/or high-viscosity therapeutic solutions can be formed into liquid droplets, which are then dispersed in a carrier liquid of a lower viscosity. Accordingly, methods for producing an injectable composition comprising a high-concentration and/or high-viscosity active agent solution are provided herein. For example, the method of producing an injectable composition comprising a high-concentration active agent solution can comprise forming one or more embodiments of the compositions or emulsions described herein. Thus, in one aspect, an injectable composition comprising an emulsion with a high concentration of an active agent can be produced. In another aspect, an injectable composition comprising an emulsion with a high viscosity active agent can be produced.

As described earlier, the injectability of the injectable compositions described herein can be characterized by injection glide force as defined herein. In some embodiments, the injection glide force of the injectable compositions described herein can be about 20 newtons or less. In some embodiments, the injection glide force of the injectable compositions described herein can be about 15 newtons or less. In some embodiments, the injection glide force of the injectable compositions described herein can be from about 2 newtons to about 20 newtons. In some embodiments, the injection glide force of the injectable compositions described herein can be from about 2 newtons to about 15 newtons.

In some embodiments of this aspect and other aspects described herein, the droplets can be microdroplets as defined herein.

In some embodiments of this aspect and other aspects described herein, the emulsion can be a single emulsion. As used herein, the term “single emulsion” refers to droplets of a single liquid phase (single-phase droplets) dispersed in a carrier liquid.

In some embodiments of this aspect and other aspects described herein, the emulsion can be a double or higher-order multiple emulsion. As used herein, the term “double or higher-order multiple emulsion” refers to droplets with at least two or more immiscible liquid phases dispersed in a carrier liquid. Stated another way, a “double or higher-order multiple emulsion” can refer to one or more larger droplets that contain one or more smaller droplets therein, and the larger droplets are dispersed in a carrier liquid.

Methods for forming different types of emulsions are known in the art and can be used or scaled up to produce the injectable compositions described herein. Example methods that can be used or scaled up to produce emulsions, emulsion-based polymer capsules, or compositions described herein include, but are not limited to high pressure homogenization, mechanical shaking, microfluidization, droplet-based microfluidics, phase inversion temperature technique, solvent displacement method, phase inversion composition method, bulk emulsification (e.g., ultrasonic emulsification and homogenization), step emulsification, membrane emulsification, parallelization of microfluidic dropmakers, any art-recognized emulsification methods, and a combination of two or more methods thereof.

In some embodiments, the emulsion can be formed using microfluidic technology. For example, an active-agent comprising first liquid can flow through a microchannel on to an impingement area resulting in droplets or microdroplets, followed by dispersion of resulting droplets or microdroplets in a carrier liquid to yield an emulsion. By way of example only, microfluidic droplet fabrication methods can be used to form droplets of a high viscosity active agent solution in a biocompatible carrier liquid (e.g., an oil-based liquid).

In some embodiments, a two phase aqueous microdroplet approach for generating droplets using two different aqueous liquids that differ in their miscibility can be used. For example, the two aqueous liquids can each comprise at least one or more incompatible polymers to create an interfacial tension between the two liquid phases that is sufficient to form droplets. In these embodiments, the droplets are formed as a water-in-water emulsion.

High-throughput methods and devices for microfluidic fabrication of droplets can also be used to produce the emulsions and/or compositions described herein. As an example, International Patent Publication No. WO 2014/186440 by Weitz et al., the content of which is incorporated herein by reference in its entirety, describes systems and methods for creating droplets by flowing a liquid from a first channel to a second channel through a plurality of side/connecting channels. The liquid exiting the side/connecting channels into the second channel can form a plurality of droplets simultaneously, thus producing droplets at a high production rate. Similar methods and devices can also be used to produce double or higher-order multiple emulsions. For example, formation of double or higher-order multiple emulsions can be achieved by forming multiple emulsions through a direct, synchronized production method and/or through the formation of a single emulsion that is collected and re-injected into a second microfluidic device to form double emulsions.

Formation of emulsions and multiple emulsions containing droplets, e.g., with a uniform size, shape, and/or a uniform number of smaller droplets contained within larger droplets is known in the art and can be used to produce the emulsions and/or compositions described herein. For example, International Patent Publication No. WO 2008/121342 by Weitz et al., the content of which is incorporated herein by reference in its entirety, describes the use of microfluidic systems to produce multiple emulsions containing uniformly sized larger droplets each containing smaller droplets. Generally, in these systems, multiple emulsions can be formed by nesting multiple immiscible liquids within a microfluidic conduit system. The multiple emulsions can be produced by first producing one or more droplets of a first liquid within a second liquid at the exit of a first conduit or microchannel. These droplets can then be transported to the end of a second conduit or microchannel, where a multiple emulsion is formed in which the second liquid surrounds the droplets of the first liquid.

In addition, formation of multiple emulsions in which the first and second droplets are formed simultaneously is known in the art and can be used to produce the emulsions and/or compositions described herein. For example, International Patent Publication Number WO 2006/096571 by Weitz et al., the content of which is incorporated herein by reference in its entirety, includes a description of various microfluidic systems in which liquids can be transported through two nested conduits or microchannels contained within another conduit or microchannel to produce multiple emulsions. Multiple conduits or microchannels are typically used in these systems, and in some cases, an inner conduit is nested within a surrounding conduit such that the exit opening of the inner conduit extends past the exit opening of the surrounding conduit. As another example, International Patent Publication Number WO 2011/028764, by Weitz et al., the content of which is incorporated herein by reference in its entirety, describes the formation of multiple emulsions, but in various systems that include certain intersections of different conduits. As a further example, International Patent Publication Number WO 2013/006661, by Weitz et al., the content of which is incorporated herein by reference in its entirety, describes another method for formation of multiple emulsions, but in various systems that are operated under a jetting flow regime.

In some embodiments, double emulsions can be generated by a microfluidic device, for example, as described in Pessi et al., International Journal of Pharmaceutics (2014) 472: 82-87, the content of which is incorporated herein by reference in its entirety. The microfluidic device generally employs a biphasic flow to produce microcapsules from double emulsion droplets with ultrathin shells. In some embodiments, the microfluidic device described in Pessi et al. (2014) can be used to prepare encapsulated droplet structures (e.g., capsules or microcapsules) with a water core, a biocompatible oil shell, and a water continuous phase. The core can comprise a high-concentration and/or high-viscosity active agent solution with any optional additives added to ensure stability. The shell can comprise oil or a polymer matrix that can not only ensure encapsulation, but can also control the release of the active agent to enable extended or delayed release, if desired.

In alternative embodiments where the shell comprise a polymer and a solvent that can be subsequently evaporated, the methods of various aspects described herein can further comprise removing or evaporating a solvent from the shell, e.g., by air-drying. Thus, droplets comprising an active-agent comprising liquid core, and a polymeric shell surrounding the core, can be produced.

Manufacture methods for polymer capsules are known in the art, including, e.g., but not limited to, spray drying, extrusion, interfacial polymerization, coacervation, layer-by-layer deposition, a combination of two or more thereof, and can be used or scaled up to produce the droplets described herein.

In some embodiments, release of active agent(s) from the droplets (e.g., double emulsion droplets) described herein can be adjusted, e.g., by selecting an appropriate liquid phase that forms the shell or outer layer of the droplets. For example, for a water-oil-water emulsion, release of active agent(s) can occur through diffusion of encapsulated active agent(s) through the oil phase. The release rate can be tuned by selection of appropriate oil (e.g., diffusion through plant oil layer is typically faster than through mineral oil layer). As another example where droplets comprise polymeric shells, release of active agent(s) can be triggered and/or accelerated by one or more stimuli. Examples of stimuli include, but are not limited to, pH, osmotic stress, enzymatic degradation, swelling, ultrasound, light, mechanical stress, and a combination of two or more thereof.

Depending on the methods used to produce an emulsion and/or desired size or size distribution of the droplets formed in an emulsion, in some embodiments, the methods of various aspects described herein can further comprise reducing the droplets into smaller droplets. In one embodiment, the droplets can be reduced into microdroplets. For example, larger droplets can be first produced by any methods known in the art (e.g., by mechanical shaking) and then passed through a series of obstructions (e.g., microposts having a cross-section of any shape, including, e.g., but not limited to, substantially rectangular, substantially square, and/or substantially circular shape) in a microchannel to break up the larger droplets into smaller ones. As an example, International Patent Publication Number WO 2014/138154, by Weitz et al., the content of which is incorporated herein by reference in its entirety, describes devices and methods for reducing or dividing droplets using a device comprising a microfluidic channel with a two-dimensional array of obstructions disposed therein. The devices and methods disclosed in the '154 application can also be used to produce a relatively monodisperse population of droplets from a polydisperse population of droplets. Accordingly, in some embodiments, the methods of various aspects described herein can further comprise converting a polydisperse population of droplets into a relatively monodisperse population of droplets.

In some embodiments, the methods of various aspects described herein can further comprise fusing droplets to form combined droplets. Methods and microfluidic devices for fusing droplets are known in the art and can be used to make the emulsions and/or compositions described herein. As an example, International Patent Publication Number WO 2014/201196, by Weitz et al., the content of which is incorporated herein by reference in its entirety, describes devices and methods for fusing droplets in a microfluidic device. In one embodiment, the method comprises (a) flowing a first droplet and a second droplet in a liquid within a microfluidic channel, wherein the first droplet and the second droplet each stabilized in the liquid using a surfactant; and (b) exposing the first droplet and/or the second droplet to a solvent that is able to alter interfacial tension of the surfactant. Examples of such solvent include, but are not limited to an alcohol, a fluorinated alcohol, a butanol, a propanol, a pentanol, a hexanol, or a combination of two or more thereof. Thus, the first droplet and the second droplet can be caused to merge into a combined droplet in the microfluidic channel.

If desirable, the droplets in an emulsion can be transferred to a different carrier liquid and/or concentrated in either the same or a different biocompatible carrier liquid. For example, the droplets in an emulsion can be concentrated to increase the number of droplets in a certain volume of a carrier liquid; thus, increasing the total amount of an active agent present in a certain volume of the carrier liquid. Accordingly, in some embodiments, the methods of various aspects described herein can further comprise separating the droplets from the emulsion. The droplets can be separated from the emulsion or carrier liquid using any methods known in the art without causing droplet breakage. For example, the separating can be performed by physical separation, e.g., but not limited to, centrifugation, size exclusion (e.g., microfluidic size exclusion, pore size exclusion), filtration, and a combination of two or more thereof.

In some embodiments, the methods of various aspects described herein can further comprise re-dispersed the separated droplets in the same or a different carrier liquid. When the separated droplets are re-dispersed in a smaller volume of a carrier liquid, a more concentrated emulsion is produced. In some embodiments, the concentrated emulsion can comprise active agent-comprising droplets up to about 60% of the total emulsion volume. Thus, high concentration/high viscosity active agent solutions can be effectively transformed into a low viscosity solution (e.g., using a low-viscosity carrier liquid) and easily injected through a needle for subcutaneous delivery.

Methods to store emulsions are known in the art and can be applied to store the droplets and/or compositions described herein. In some embodiments, emulsion of droplets and/or compositions described herein can be stored at temperatures ranging between 0° C. and 30° C. In some embodiments, the final solution of concentrated emulsions can be prepared through addition of water to continuous phase, and the emulsions or droplets can then be freeze-dried.

Emulsions or injectable compositions produced by the methods of making described herein are also encompassed by the scope of various aspects described herein.

Articles of Manufacture (e.g., Containers and Injections Devices) for Parenteral Administration of High-Concentration and/or High-Viscosity Active Agents

The compositions described herein (including emulsions and injectable compositions) can be packaged in a container or device, e.g., for parenteral administration. In some embodiments, the compositions described herein can be packaged, e.g., in container. Accordingly, another aspect provided herein is a container comprising one or more embodiments of the compositions, emulsions or injectable compositions described herein. In some embodiments, the container can be a vial, a dual-chamber vial, a bottle, or a test tube.

A label or a package insert indicating directions for use can also be adhered on the container or associated with the container.

In some embodiments, the compositions described herein can be loaded into an injection device. Accordingly, described herein is also an injection device comprising (i) a chamber and (ii) one or more embodiments of the compositions, emulsions, or injectable compositions disposed in the chamber.

In some embodiments, the injection device can further comprise a needle adaptably coupled to the chamber. The needle can have a gauge of at least 18 or above, including, e.g., at least 19, at least 20, at least 21, at least 22, at least 23, at least 24, at least 25, at least 26, at least 27, at least 28, at least 29, at least 30, at least 31, at least 32, at least 33, or at least 34. In these embodiments, the droplets of the compositions, emulsions, or injectable compositions described herein can have a dimension that is smaller than the inner diameter of the needle. Thus, the compositions, emulsions, or injectable compositions can be injected quickly, while an active agent at a high concentration can still be delivered in a rapid manner.

In some embodiments, the injection device can be an autoinjector. In some embodiments, the injection device can be a prefilled syringe. In some embodiments, the chamber can comprise a syringe barrel.

The pre-filled containers and injection described herein can be used for parenteral administration. As used herein, the term “parenteral administration” includes subcutaneous, intradermal, intramuscular, intravenous, intrathecal, and intraarticular administration. In some embodiments, the pre-filled containers and injection described herein can be used for subcutaneous administration.

### Methods of Use

The compositions, emulsions, and/or articles described herein can be used in various applications (e.g., but not limited to, plastic surgery, tissue reconstruction, medical treatment, manufacturing, cosmetic and/or skincare products, food and beverages, and construction), where a high-concentration and/or high viscosity of an active agent composition is desired to be delivered to a target site or area, e.g., through a small orifice such as an injection needle, a catheter, or a tubing.

In some embodiments, the compositions, emulsions, and articles described herein can be used to administer a high concentration dose of an active agent to a subject in need thereof, e.g., to treat a disease or disorder. Thus, methods of administering to a subject a high concentration dose of an active agent, e.g., for treatment of a disease or disorder, are also provided herein. The method comprises injecting the subject with one or more embodiments of the compositions or emulsions described herein, or using one or more embodiments of the articles (e.g., pre-filled containers and/or injection devices) described herein.

Given the high concentration of an active agent in the compositions and/or emulsions described herein, smaller volumes of the compositions (including, e.g., pharmaceutical compositions, and injectable compositions) and emulsions described herein can be administered to a patient, while maintaining the efficacy as compared to conventionally available preparations having a lower active agent concentration.

While the compositions, emulsions, and articles described herein can be used in parenteral administration of an active agent, they can be more beneficial when used for subcutaneous administration where the injection volume is usually small, e.g., no more than 2 mL. Thus, in some embodiments, the injection can be performed by subcutaneous administration. In some embodiments, the injection volume of the high-concentration active agent dose can be no more than 1.5 mL. In some embodiments, the injection volume of the high-concentration active agent dose can be no more than 1.0 mL.

Despite its relatively high concentration, the compositions (including, e.g., pharmaceutical compositions, and injectable compositions) and emulsions described herein can be administered in a fast and simple manner due to its overall low viscosity. In particular, conventional means currently used for subcutaneous administration can be employed to administer the compositions (including, e.g., pharmaceutical compositions, and injectable compositions) and emulsions described herein. For example, administration by direct manual push from a syringe is sufficient. The capability of using simple devices, such as conventional syringes, to administer the compositions (including, e.g., pharmaceutical compositions, and injectable compositions) and emulsions described herein can increase the acceptance of subcutaneous administration and ultimately lowers the cost of the treatment regimen.

Pharmaceutical Compositions Comprising the Compositions and/or Emulsions Described Herein

In some embodiments, the compositions and/or emulsions described herein can be formulated for administration in vivo, and are thus provided in pharmaceutically acceptable compositions. As used herein, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The pharmaceutically acceptable composition can further comprise one or more pharmaceutically acceptable carriers (additives) and/or diluents. As used herein, the term “pharmaceutically-acceptable carrier” means a pharmaceutically-acceptable material, composition or vehicle, such as a liquid, diluent, excipient, manufacturing aid or encapsulating material, for administration of the compositions or emulsions described herein. Each carrier must be “acceptable” in the sense of being compatible with the other ingredients of the formulation and not injurious to the patient. Pharmaceutically acceptable carriers include any and all solvents, dispersion media, coatings, antibacterial and antifungal agents, isotonic and absorption delaying agents, and the like which are compatible with the activity of the compositions or emulsions described herein and are physiologically acceptable to the subject. Some examples of materials which can serve as pharmaceutically-acceptable carriers include: (i) sugars, such as lactose, glucose and sucrose; (ii) starches, such as corn starch and potato starch; (iii) cellulose, and its derivatives, such as sodium carboxymethyl cellulose, methylcellulose, ethyl cellulose, microcrystalline cellulose and cellulose acetate; (iv) powdered tragacanth; (v) malt; (vi) gelatin; (vii) lubricating agents, such as magnesium stearate, sodium lauryl sulfate and talc; (viii) excipients, such as cocoa butter and suppository waxes; (ix) oils, such as peanut oil, cottonseed oil, safflower oil, sesame oil, olive oil, corn oil and soybean oil; (x) glycols, such as propylene glycol; (xi) polyols, such as glycerin, sorbitol, mannitol and polyethylene glycol (PEG); (xii) esters, such as ethyl oleate and ethyl laurate; (xiii) agar; (xiv) buffering agents, such as magnesium hydroxide and aluminum hydroxide; (xv) alginic acid; (xvi) pyrogen-free water; (xvii) isotonic saline; (xviii) Ringer's solution; (xix) ethyl alcohol; (xx) pH buffered solutions; (xxi) polyesters, polycarbonates and/or polyanhydrides; (xxii) bulking agents, such as polypeptides and amino acids (xxiii) serum component, such as serum albumin, HDL and LDL; (xxiv) C2-C12 alcohols, such as ethanol; and (xxv) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it may be desirable to include isotonic agents, for example, sugars, sodium chloride, and the like.

The compositions can also contain auxiliary substances such as wetting or emulsifying agents, pH buffering agents, gelling or viscosity enhancing additives, preservatives, colors, and the like, depending upon the route of administration and the preparation desired. Standard texts, such as “REMINGTON′S PHARMACEUTICAL SCIENCE”, 17th edition, 1985, incorporated herein by reference, may be consulted to prepare suitable preparations, without undue experimentation. With respect to the pharmaceutical compositions described herein, however, any vehicle, diluent, or additive used should have to be biocompatible or inert with the compositions and/or emulsions described herein and/or active agents described herein.

The compositions can be isotonic, i.e., they can have the same osmotic pressure as blood and lacrimal fluid. The desired isotonicity of the pharmaceutical compositions described herein can be accomplished using sodium chloride, or other pharmaceutically acceptable agents such as dextrose, boric acid, sodium tartrate, propylene glycol or other inorganic or organic solutes. In one embodiment, sodium chloride is used in buffers containing sodium ions. Isotonicity can be measured using a vapor pressure or ice-freezing type osmometer, for example.

Viscosity of the pharmaceutical compositions can be maintained at the selected level using a pharmaceutically acceptable thickening agent. For example, while not necessary, a pharmaceutically acceptable thickening agent can be added to a carrier liquid as described herein to achieve the selected viscosity. In one embodiment, methylcellulose is used because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

Typically, any additives (in addition to the active agents described herein) can be present in an amount of 0.001 to 50 wt % solution in phosphate buffered saline.

It will be appreciated that the exact dosage of the composition is chosen by the individual physician in view of the patient to be treated. In general, dosage and administration are adjusted to provide an effective amount of the composition to the patient being treated. As used herein, the “effective amount” of a composition refers to the amount necessary to elicit the desired biological response. As will be appreciated by those of ordinary skill in this art, the effective amount of a composition may vary depending on such factors as the desired biological endpoint, the drug to be delivered, the target tissue, the route of administration, etc. For example, the effective amount of the composition containing an anti-cancer drug might be the amount that results in a reduction in tumor size by a desired amount over a desired period of time. Additional factors which may be taken into account include the severity of the disease state; age, weight and gender of the patient being treated; diet, time and frequency of administration; drug combinations; reaction sensitivities; and tolerance/response to therapy.

The compositions can be formulated in dosage unit form for ease of administration and uniformity of dosage. The expression “dosage unit form” as used herein refers to a physically discrete unit of composition appropriate for the patient to be treated. It will be understood, however, that the total daily usage of the compositions described herein will be decided by the attending physician within the scope of sound medical judgment. For any composition, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. Therapeutic efficacy and toxicity of composition can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and it can be expressed as the ratio, LD50/ED50. Pharmaceutical compositions which exhibit large therapeutic indices may be useful in some embodiments. The data obtained from cell culture assays and animal studies can be used in formulating a range of dosage for human use.

The compositions of various aspects described herein can be administered to a human patient in accord with known methods, such as intravenous administration, e.g., as a bolus or by continuous infusion over a period of time, by intramuscular, intraperitoneal, intracerobrospinal, subcutaneous, intra-articular, intrasynovial, or intrathecal administration. In some embodiments, the compositions of various aspects described herein can be administered by intramuscular or subcutaneous methods. In some embodiments, the compositions of various aspects described herein can be administered to a subject (e.g., a human patient) by oral, aerosol, occular or transdermal administration.

Without wishing to bebound by a theory, the methods and compostions of the invention can provide stability and/or protection for high concentration biologics or other therapeutic formualtions against stomach acid when such high concentration biologics or other therapeutic formualtions are administered orally. Further, the methods and compostions of the invention are useful in aerosol delivery and provide control over sustain release of high concentration biologics or other therapeutic formualtions.

In some embodiments, the compositions of various aspects described herein can be administered by subcutaneous delivery. For subcutaneous delivery, the compositions can be administered via syringe (e.g. pre-filled syringe); autoinjector; injection device (e.g. the INJECT-EASE™ and GENJECT™ device); injector pen (such as the GENPEN™); or other device suitable for administering a composition described herein subcutaneously. In one embodiment, the compositions of various aspects described herein can be administered by a pre-filled syringe.

Exemplary Active Agents That can be Encapsulated in Droplets of the Compositions and/or Emulsions Described Herein

Any active agent can be encapsulated in the droplets of the compositions and emulsions described herein and/or using the droplet fabrication methods described herein. As used herein, the term “active agent” refers to an active ingredient that is intended for use in a particular application. In some embodiments, the term “active agent” refers to an agent that possesses therapeutic, prophylactic, or diagnostic properties in vivo, for example when administered to a human subject or an animal, including mammal and domestic animals, e.g., pet animals, including, e.g., cats and dogs. Examples of active agents include, but are not limited to, proteins, peptides, antibodies, growth factors, nucleic acids, sugars, antigens, vaccines, enzymes, cells, small molecules such as antibiotics, steroids, decongestants, anesthetics, sedatives, and a combination of two or more thereof.

In some embodiments, the active agent to be encapsulated in the compositions and/or emulsions described herein can comprise an organic molecule such as a drug, peptide, protein, carbohydrate (including monosaccharides, oligosaccharides, and polysaccharides), nucleoprotein, mucoprotein, lipoprotein, synthetic polypeptide or protein, or a small molecule linked to a protein, glycoprotein, steroid, nucleic acid (any form of DNA, including cDNA, or RNA, or a fragment thereof), nucleotide, nucleoside, oligonucleotides (including antisense oligonucleotides), gene, lipid, hormone, vitamin, including vitamin C and vitamin E, or combination thereof.

In some embodiments, the active agent to be encapsulated in the compositions and/or emulsions described herein can comprise a therapeutic active agent. Examples of therapeutic active agents include, but are not limited to, immunosuppressants, antioxidants, anesthetics, chemotherapeutic agents, steroids (including retinoids), hormones, antibiotics, antivirals, antifungals, antiproliferatives, antihistamines, anticoagulants, antiphotoaging agents, melanotropic peptides, nonsteroidal and steroidal anti-inflammatory compounds, antipsychotics, and radiation absorbers, including UV-absorbers. Other non-limiting examples of active agents include anti-infectives such as nitrofurazone, sodium propionate, antibiotics, including penicillin, tetracycline, oxytetracycline, chlorotetracycline, bacitracin, nystatin, streptomycin, neomycin, polymyxin, gramicidin, chloramphenicol, erythromycin, and azithromycin; sulfonamides, including sulfacetamide, sulfamethizole, sulfamethazine, sulfadiazine, sulfamerazine, and sulfisoxazole, and anti-virals including idoxuridine; antiallergenics such as antazoline, methapyritene, chlorpheniramine, pyrilamine prophenpyridamine, hydrocortisone, cortisone, hydrocortisone acetate, dexamethasone, dexamethasone 21-phosphate, fluocinolone, triamcinolone, medrysone, prednisolone, prednisolone 21-sodium succinate, and prednisolone acetate; desensitizing agents such as ragweed pollen antigens, hay fever pollen antigens, dust antigen and milk antigen; decongestants such as phenylephrine, naphazoline, and tetrahydrazoline; miotics and anticholinesterases such as pilocarpine, esperine salicylate, carbachol, diisopropyl fluorophosphate, phospholine iodide, and demecarium bromide; parasympatholytics such as atropine sulfate, cyclopentolate, homatropine, scopolamine, tropicamide, eucatropine, and hydroxyamphetamine; sympathomimetics such as epinephrine; sedatives and hypnotics such as pentobarbital sodium, phenobarbital, secobarbital sodium, codeine, (a-bromoisovaleryl) urea, carbromal; psychic energizers such as 3-(2-aminopropyl) indole acetate and 3-(2-aminobutyl) indole acetate; tranquilizers such as reserpine, chlorpromayline, and thiopropazate; androgenic steroids such as methyl-testosterone and fluorymesterone; estrogens such as estrone, 17-β-estradiol, ethinyl estradiol, and diethyl stilbestrol; progestational agents such as progesterone, megestrol, melengestrol, chlormadinone, ethisterone, norethynodrel, 19-norprogesterone, norethindrone, medroxyprogesterone and 17-β-hydroxy-progesterone; humoral agents such as the prostaglandins, for example PGE1, PGE2 and PGF2; antipyretics such as aspirin, sodium salicylate, and salicylamide; antispasmodics such as atropine, methantheline, papaverine, and methscopolamine bromide; antimalarials such as the 4-aminoquinolines, 8-aminoquinolines, chloroquine, and pyrimethamine, antihistamines such as diphenhydramine, dimenhydrinate, tripelennamine, perphenazine, and chlorphenazine; cardioactive agents such as dibenzhydroflume thiazide, flumethiazide, chlorothiazide, and aminotrate; nutritional agents such as vitamins, natural and synthetic bioactive peptides and proteins, including growth factors, cell adhesion factors, cytokines, and biological response modifiers.

In some embodiments, the active agent to be encapsulated in droplets of the compositions and/or emulsions described herein can comprise a vaccine. As used herein, the term “vaccine” refers to a composition comprising at least one antigen or immunogen in a pharmaceutically acceptable vehicle useful for inducing an immune response in a host. The antigen can be derived from a cell, bacteria, or virus particle, or portion thereof. An antigen can be a protein, peptide, polysaccharide, glycoprotein, glycolipid, nucleic acid, or a combination of two or more thereof, which elicits an immunogenic response in a human subject or an animal, for example, a mammal, bird, or fish. The immunogenic response can be humoral or cell-mediated. In some embodiments where the material to which the immunogenic response is to be directed is poorly antigenic, it can be conjugated to a carrier, such as albumin, or to a hapten, using standard covalent binding techniques known in the art, for example, with one of the several commercially available reagent kits. Examples of antigens include, but are not limited to, viral proteins such as influenza proteins, human immunodeficiency virus (HIV) proteins, and hepatitis A, B, or C proteins, and bacterial proteins, lipopolysaccharides such as gram negative bacterial cell walls and Neisseria gonorrhea proteins, parvovirus, and a combination of two or more thereof.

Agents such as insecticides, pesticides, fungicides, rodenticides, plant nutrients, and growth promoters also can be encapsulated in droplets of the compositions and/or emulsions described herein.

In some embodiments, the active agent to be encapsulated in droplets of the compositions and/or emulsions described herein can comprise an antibody and/or an antibody fragment. The term “antibody” or “antibodies” as used herein refers to immunoglobulin molecule(s) and immunologically active portions of immunoglobulin molecule(s) (molecules that contain an antigen binding site which specifically binds to an antigen), including monoclonal antibodies (including full length monoclonal antibodies), polyclonal antibodies, multispecific antibodies (for example, bispecific antibodies), chimeric antibodies, humanized antibodies, human antibodies, and single chain antibodies (scFvs).

As used herein, the term “antibody fragment,” as used herein, refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H1} domains; (ii) the Fab′ fragment, which is a Fab fragment having one or more cysteine residues at the C-terminus of the C_{H1} domain; (iii) the Fd fragment having V_{H} and C_{H1} domains; (iv) the Fd′ fragment having V_{H} and C_{H1} domains and one or more cysteine residues at the C-terminus of the C_{H1} domain; (v) the Fv fragment having the V_{L} and V_{H} domains of a single arm of an antibody; (vi) the dAb fragment (Ward et al., Nature 341, 544-546 (1989)) which consists of a V_{H} domain; (vii) isolated CDR regions; (viii) F(ab′)₂ fragments, a bivalent fragment including two Fab′ fragments linked by a disulfide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H1}-V_{H}-C_{H1}) which, together with complementary light chain polypeptides, form a pair of antigen binding regions (Zapata et al. Protein Eng. 8(10):1057-1062 (1995); and U.S. Pat. No. 5,641,870).

In some embodiments, the active agent to be encapsulated in droplets of the compositions and/or emulsions described herein can comprise a single-domain antibody. By the term “single-domain antibody” or “sdAb”, it is meant an antibody fragment comprising a single protein domain. Single domain antibodies can comprise any variable fragment, including V_{L}, V_{H}, V_{HH}, and V_{NAR}, and can be naturally-occurring or produced by recombinant technologies. For example, V_{H}, V_{L}, V_{HH}, and V_{NAR} domains can be generated by techniques well known in the art (Holt, et al., 2003; Jespers, et al., 2004a; Jespers, et al., 2004b ; Tanha, et al., 2001; Tanha, et al., 2002; Tanha, et al., 2006; Revets, et al., 2005; Holliger, et al., 2005; Harmsen, et al., 2007; Liu, et al., 2007; Dooley, et al., 2003; Nuttall, et al., 2001; Nuttall, et al., 2000; Hoogenboom, 2005; Arbabi-Ghahroudi et al., 2008). In the recombinant DNA technology approach, libraries of sdAbs can be constructed in a variety of ways, “displayed” in a variety of formats such as phage display, yeast display, ribosome display, and subjected to selection to isolate binders to the targets of interest (panning). Examples of libraries include immune libraries derived from llama, shark or human immunized with the target antigen; non-immune/naive libraries derived from non-immunized llama, camel, shark or human; or synthetic or semi-synthetic libraries such as V_{H}, V_{L}, V_{HH} or V_{NAR} libraries. In one embodiment, the sdAb can be a heavy variable domain (V_{H}).

In some embodiments, the active agent to be encapsulated in droplets of the compositions and/or emulsions described herein can comprise a nanobody. A nanobody (Nb) is single variable domain (V_{H}H) of a naturally occurring single-chain antibody and is known to the person skilled in the art. They are generally derived from heavy chain only antibodies, for example, in camelids and sharks. The term “Camelids” refers to old world camelids (Camelus bactrianus and Camelus dromedarius) and new world camelids (for example, Lama paccos, Lama glama, Lama guanicoe and Lama vicugna). The small size and unique biophysical properties of Nbs exceed conventional antibody fragments for the recognition of uncommon or hidden epitopes and for binding into cavities or active sites of protein targets. Further, Nbs can be designed as multi-specific and multivalent antibodies or attached to reporter molecules. Nbs can survive the gastro-intestinal system and can easily be manufactured. Therefore, Nbs can be used in many applications including drug discovery and therapy, but also as a versatile and valuable tool for purification, functional study and crystallization of proteins.

The nanobodies generally comprise a single amino acid chain that can be considered to comprise our “framework regions” or FRs and three “complementarity determining regions” or CDRs. The term “complementarity determining region” or “CDR” refers to variable regions in nanobodies and contains the amino acid sequences capable of specifically binding to antigenic targets. These CDR regions account for the basic specificity of the nanobody for a particular antigenic determinant structure. Such regions are also referred to as “hypervariable regions.” The nanobodies have three CDR regions, each non-contiguous with the others (termed CDR1, CDR2, CDR3).

In some embodiments, the active agent to be encapsulated in droplets of the compositions and/or emulsions described herein can comprise a monoclonal antibody. The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigen. Furthermore, in contrast to polyclonal antibody preparations that typically include different antibodies directed against different determinants (epitopes), each monoclonal antibody is directed against a single determinant on the antigen. The modifier “monoclonal” is not to be construed as requiring production of the antibody by any particular method. For example, the monoclonal antibodies to be used in accordance with various aspects described herein can be made by the hybridoma method first described by Kohler et al., Nature 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature 352:624-628 (1991) or Marks et al., J. Mol. Biol. 222:581-597 (1991), for example.

In some embodiments, the active agent to be encapsulated in droplets of the compositions and/or emulsions described herein can comprise “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al, Proc. Natl. Acad. Sci. USA 81:6851-6855 (1984)).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies can comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

As used herein, a “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art. In one embodiment, the human antibody is selected from a phage library, where that phage library expresses human antibodies (Vaughan et al. Nature Biotechnology 14:309-314 (1996): Sheets et al. Proc. Natl. Acad. Sci. 95:6157-6162 (1998)); Hoogenboom and Winter, J. Mol. Biol., 227:381 (1991); Marks et al., J. Mol. Biol., 222:581 (1991)). Human antibodies can also be made by introducing human immunoglobulin loci into transgenic animals, e.g., mice in which the endogenous mouse immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368:812-13 (1994); Fishwild et al., Nature Biotechnology 14: 845-51 (1996); Neuberger, Nature Biotechnology 14: 826 (1996); Lonberg and Huszar, Intern. Rev. Immunol. 13:65-93 (1995). Alternatively, the human antibody can be prepared via immortalization of human B lymphocytes producing an antibody directed against a target antigen (such B lymphocytes can be recovered from an individual or can have been immunized in vitro). See, e.g., Cole et al., Monoclonal Antibodies and Cancer Therapy, Alan R. Liss, p. 77 (1985); Boerner et al., J. Immunol., 147 (1):86-95 (1991); and U.S. Pat. No. 5,750,373.

In some embodiments, the active agent to be encapsulated in droplets of the compositions and/or emulsions described herein can comprise cells. The term “cell” used herein refers to any cell, prokaryotic or eukaryotic, including plant, yeast, worm, insect and mammalian. Mammalian cells include, without limitation; primate, human and a cell from any animal of interest, including without limitation; mouse, hamster, rabbit, dog, cat, transgenic animal domestic animals, such as equine, bovine, murine, ovine, canine, feline, etc. The cells may be a wide variety of tissue types without limitation such as; hematopoietic, neural, mesenchymal, cutaneous, mucosal, stromal, muscle spleen, reticuloendothelial, epithelial, endothelial, hepatic, kidney, gastrointestinal, pulmonary, T-cells etc. Stem cells, embryonic stem (ES) cells, ES-derived cells and stem cell progenitors are also included, including without limitation, hematopoeitic, stromal, muscle, cardiovascular, hepatic, pulmonary, gastrointestinal stem cells, etc.

In some embodiments, the active agent to be encapsulated in droplets of the compositions and/or emulsions described herein can be selected from, but is not limited to, one or a combination of two or more of the following biological agents:
Antibodies such as Adalimumab (e.g., Humira®), Blinatumimab, Brodalumab, Carfilzomib (e.g., Kyprolis®), Cetuximab, Evolocumab, Infliximab, Romosozumab, Rilotumumab, Trastuzumab, Panitumumab (e.g., Vectibix®), Denosumab, and Trebananib;
Polypeptides such as growth hormones (including human growth hormone and bovine growth hormone), thyroid stimulating hormone, anti-clotting factors such as Protein C, and growth factors such as vascular endothelial growth factor (VEGF), platelet derived growth factor (PDGF), and insulin-like growth factor-I and II (IGF-I and IGF-II); and
Other biological agents such as antibody fragments and viral antigens.

In some embodiments, the active agent to be encapsulated in droplets of the compositions and/or emulsions described herein can be an active agent described herein that is too viscous to be injected when it is administered by itself at a high concentration, e.g., at least about 50 mg/mL or higher, including, e.g., at least about 100 mg/mL. For example, the active agent when administered by itself at a high concentration is too viscous to be injected to a target site within 10 seconds or less (e.g., within 5 seconds or less).

### Some Selected Definitions

For convenience, certain terms employed herein, in the specification, examples and appended claims are collected here. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used to described the present invention, in connection with percentages means ±5%. When “0%” is used to describe the amount of a component, it is understood that this includes situations where only trace amounts of the component are present.

As used in this specification and the appended claims, the singular forms “a,” “an,” and “the” include singular and plural references unless the context clearly dictates otherwise. Thus for example, references to “the method” includes one or more methods, and/or steps of the type described herein and/or which will become apparent to those persons skilled in the art upon reading this disclosure and so forth. For example, the term “an active agent” includes reference to one or a plurality (e.g., two or more) of active agent(s) and the term “the active agent” includes reference to one or a plurality (e.g., two or more) of active agent(s) and equivalents thereof known to those skilled in the art, and so forth, it is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, “buffer” refers to a buffered solution that resists changes in pH by the action of its acid-base conjugate components. The buffer can generally have a pH from about 4.0 to about 8.0, for example from about 5.0 to about 7.0, e.g. from about 5.8 to about 6.2, and in one embodiment its pH is about 6.0. Examples of buffers that can be used to control the pH in this range include, but are not limited to, acetate, succinate, succinate, gluconate, histidine, citrate, glycylglycine and other organic acid buffers.

As used herein, the term “high-concentration active agent solution” refers to a composition comprising at least one active agent at a concentration of at least about 50 mg/mL or higher. In some embodiments, the term “high-concentration active agent solution” refers to a composition comprising at least one active agent at a concentration of at least about 75 mg/mL or higher. In some embodiments, the term “high-concentration active agent solution” refers to a composition comprising at least one active agent at a concentration of at least about 100 mg/mL or higher, including, e.g., at least about 150 mg/mL, at least about 200 mg/mL, at least about 250 mg/mL, at least about 300 mg/mL, at least about 350 mg/mL, at least about 400 mg/mL, at least about 450 mg/mL, at least about 500 mg/mL, or higher.

As used herein, the term “high-viscosity active agent solution” refers to a solution of at least one or more active agents having a viscosity of at least about 50 cP or higher. In some embodiments, the term “high-viscosity active agent solution” refers to a solution of at least one or more active agents having a viscosity of at least about 60 cP or higher, including, e.g., at least about 70 cP, at least about 80 cP, at least about 90 cP, at least about 100 cP or higher.

As used herein, the term “subject” refers to any living organism which can be administered to the pharmaceutical compositions of the present invention and in which cancer or a proliferative disorder can occur. The term includes, but is not limited to, humans, non-human primates such as chimpanzees and other apes and monkey species; farm animals such as cattle, sheep, pigs, goats and horses, domestic subjects such as dogs and cats, laboratory animals including rodents such as mice, rats and guinea pigs, and the like. The term does not denote a particular age or sex. Thus, adult and newborn subjects, as well as fetuses, whether male or female, are intended to be covered. The term “subject” is also intended to include living organisms susceptible to conditions or disease states as generally disclosed, but not limited to, throughout this specification. Examples of subjects include humans, dogs, cats, cows, goats, and mice. The term subject is further intended to include transgenic species. As used herein, the terms “subject” and “individual” are used interchangeably and are intended to refer to an animal, for example a human, to whom treatment, including prophylactic treatment, with the pharmaceutical composition according to the present invention, is provided, including, but not limited to humans and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

The term “disease” or “disorder” is used interchangeably herein, refers to any alternation in state of the body or of some of the organs, interrupting or disturbing the performance of the functions and/or causing symptoms such as discomfort, dysfunction, distress, or even death to the person afflicted or those in contact with a person. A disease or disorder can also related to a distemper, ailing, ailment, malady, disorder, sickness, illness, complaint, inderdisposion, affection.

As used herein, the terms “treat” or “treatment” or “treating” refers to both therapeutic treatment and prophylactic or preventative measures, wherein the object is to prevent or slow the development of the disease, such as slow down the development of a tumor, the spread of cancer, or reducing at least one effect or symptom of a condition, disease or disorder associated with inappropriate proliferation or a cell mass, for example cancer. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

As used interchangeably herein, the term “substantially” means a proportion of at least about 60%, or preferably at least about 70% or at least about 80%, or at least about 90%, at least about 95%, at least about 97% or at least about 99% or more, or any integer between 70% and 100%. In some embodiments, the term “essentially ” means a proportion of at least about 90%, at least about 95%, at least about 98%, at least about 99% or more, or any integer between 90% and 100%. In some embodiments, the term “essentially” can include 100%.

All patents, patent applications, and publications identified in this document are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

### EXAMPLES

The following examples are not intended to limit the scope of the invention, but are rather intended to be exemplary of certain embodiments.

### Example 1

### One Embodiment of Encapsulation of High Viscosity Solutions

Exemplary chemicals used to make droplets: Poly(vinyl alcohol) (PVA, Mw=13 000-23 000 g/mol, 98% hydrolyzed), Span 80, Tween 80 (all Aldrich), Sucrose (BDH), Mineral oil (extra heavy, Spectrum)

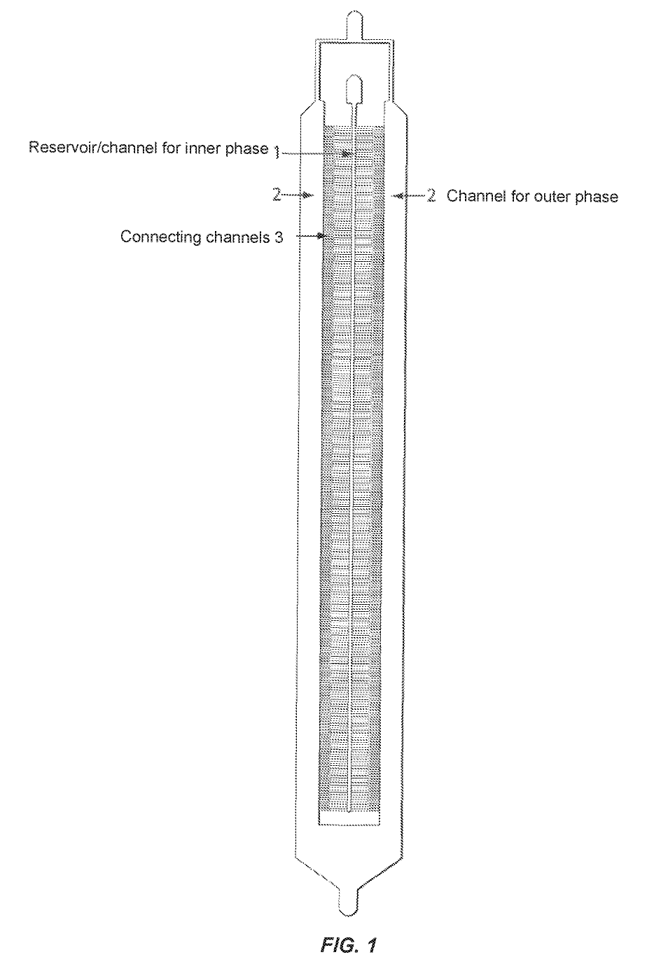

Exemplary microfluidic device used to make droplets: Millipede design in PDMS, hydrophilic polyelectrolyte coating, 300 nozzles, nozzle dimensions: 120 μm width and 20 μm height (aspect ratio 6:1). Various embodiments of the Millipede design are described in the International Patent Application No. WO 2014/186440, the contents of which are incorporated herein by reference in its entirety. In one embodiment, the Millipede design is described in Example 2 of the '440 patent application, the contents of which are incorporated herein by reference in its entirety. FIG. 1 shows an exemplary embodiment of a Millipede emulsion device.

Exemplary Methods for Fabrication of Double Emulsions:

Solution I: In one embodiment, an inner phase can comprise saturated sucrose in water and deionized water at a volume ratio of about 9:1, and about 10 wt % Tween 80. The viscosity η of such inner phase solution is about 620 cP at 25° C. In this Example, saturated sucrose solution is used to model a first liquid comprising an active agent described herein (e.g., a high-concentration active agent solution or a high viscosity solution).

Solution II: In one embodiment, a middle phase can comprise mineral oil and about 10 wt % Span 80. The viscosity η of such middle phase solution is about 134 cP at 25° C.

Solution III: In one embodiment, an outer phase can comprise water, about 10 wt % poly(vinyl alcohol), and about 75 wt % sucrose. The viscosity η of such outer phase solution is about 80 cP at 25° C.

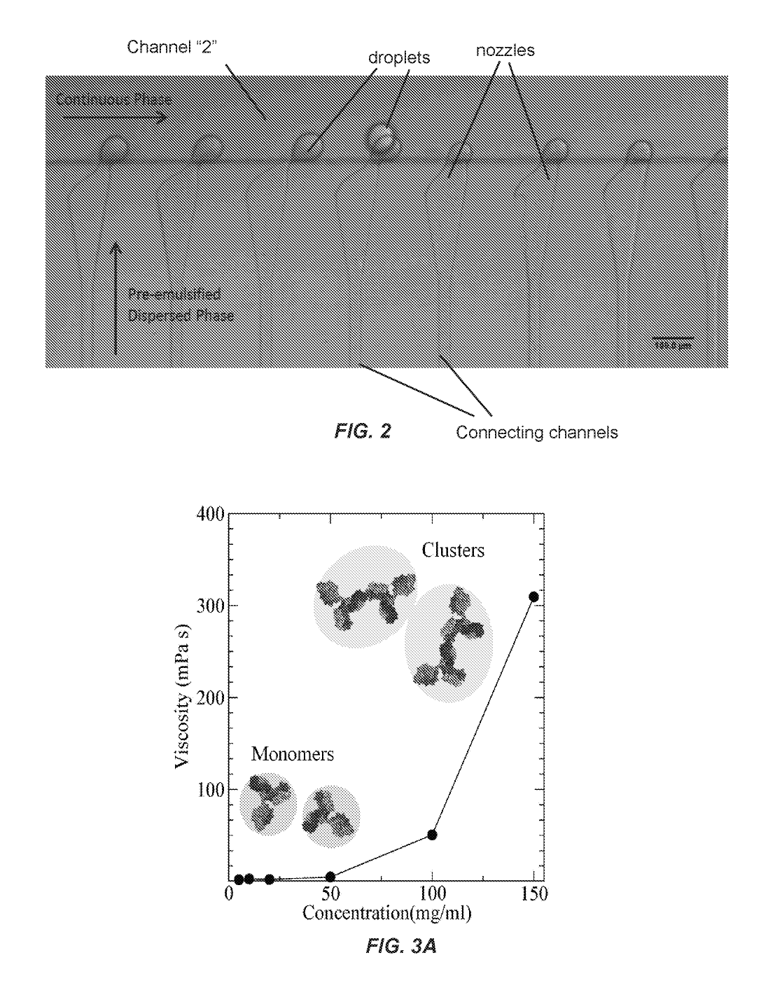

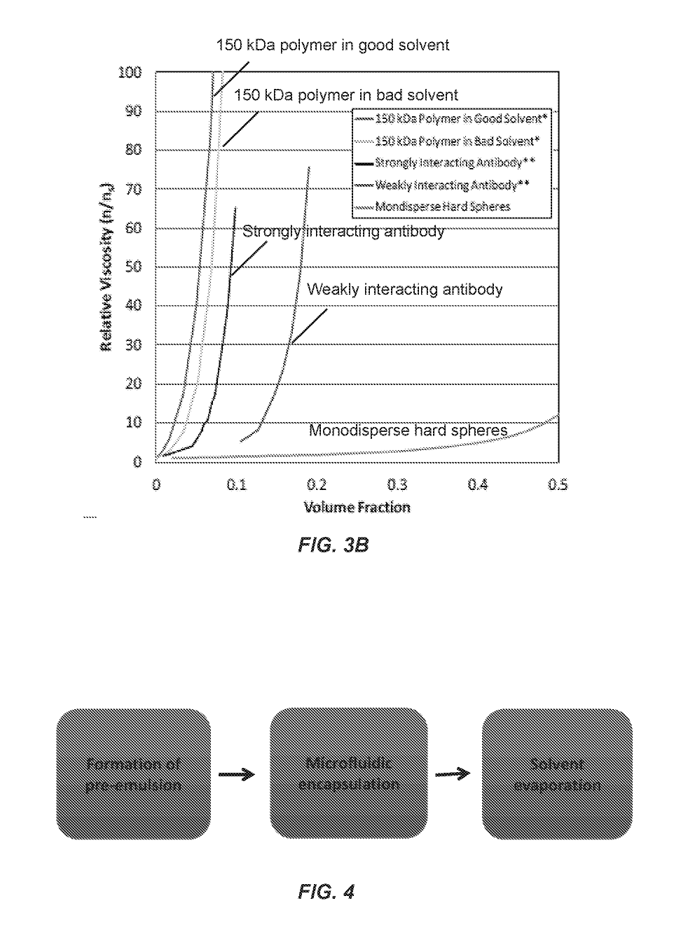

To fabricate double emulsions, Solution I (e.g., ˜1 mL) was dispersed in Solution II (e.g., ˜2 mL) by vortexing, e.g., for about one minute, to create the first emulsion, in which droplets of Solution I are dispersed in Solution II. A microfluidic Millipede single emulsion device (FIGS. 1-2) was then used to create monodisperse double emulsion droplets. The first emulsion produced by dispersion of Solution I in Solution II was used as an inner dispersed phase, while Solution III was used as a continuous phase. The inner dispersed phase was injected into a channel “1” of Millipede microfluidic device of FIG. 1 with a flow rate of about 500 μL/h and the continuous phase was injected into a channel “2” (see FIG. 1) with a flow rate of 2000 μL/h (FIGS. 1-2). The droplets comprising a core of highly viscous Solution I and a shell of Solution II surrounding the core were produced through the nozzles and dispersed in Solution III with a lower viscosity.

### Example 2

### Another Embodiment of Encapsulation of High Viscosity Solutions

In this example, an aqueous solution with a viscosity of about 600 cP was used as a model high concentration active agent solution. The encapsulation methods described herein can be used to encapsulate a solution of any viscosity and/or concentration that would be otherwise too viscous for subcutaneous injection alone by itself.

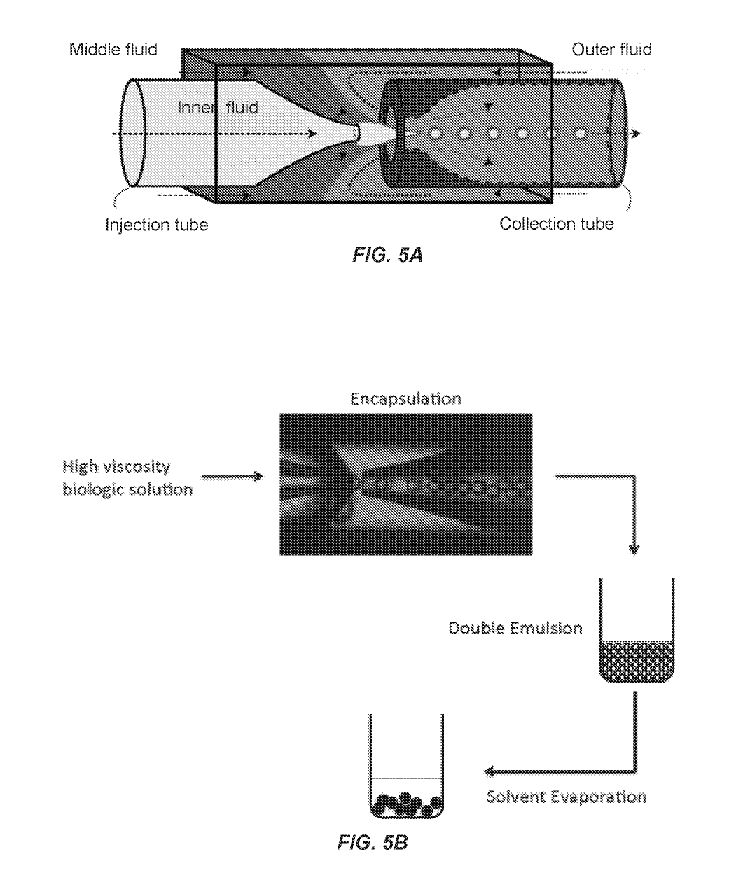

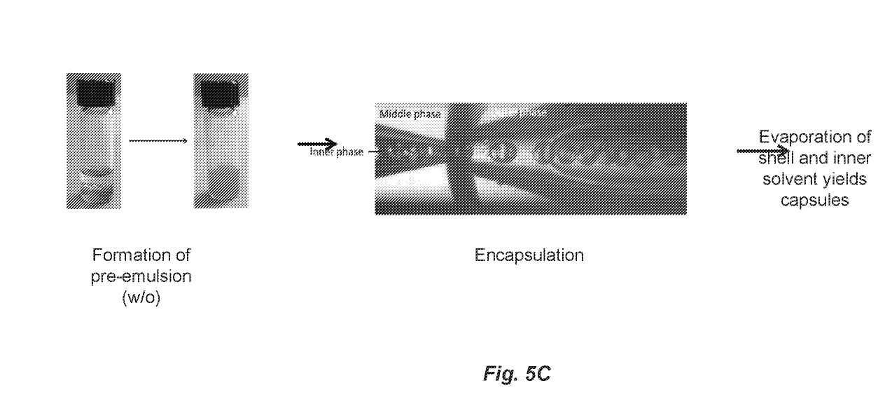

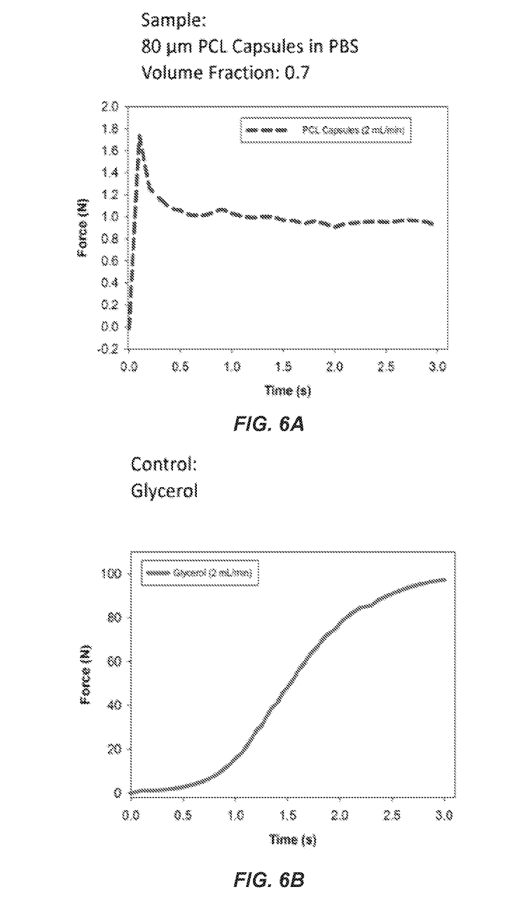

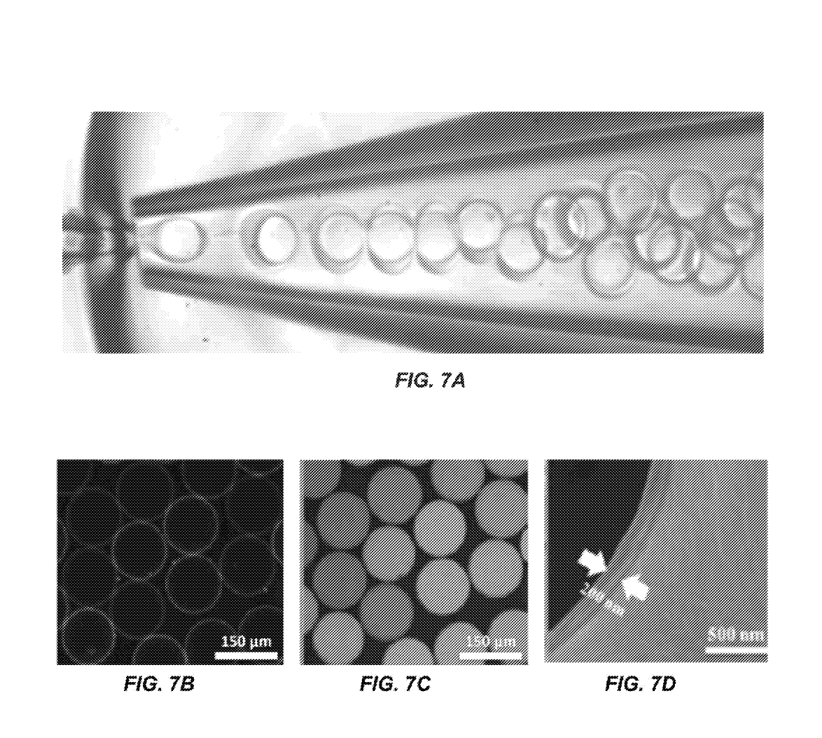

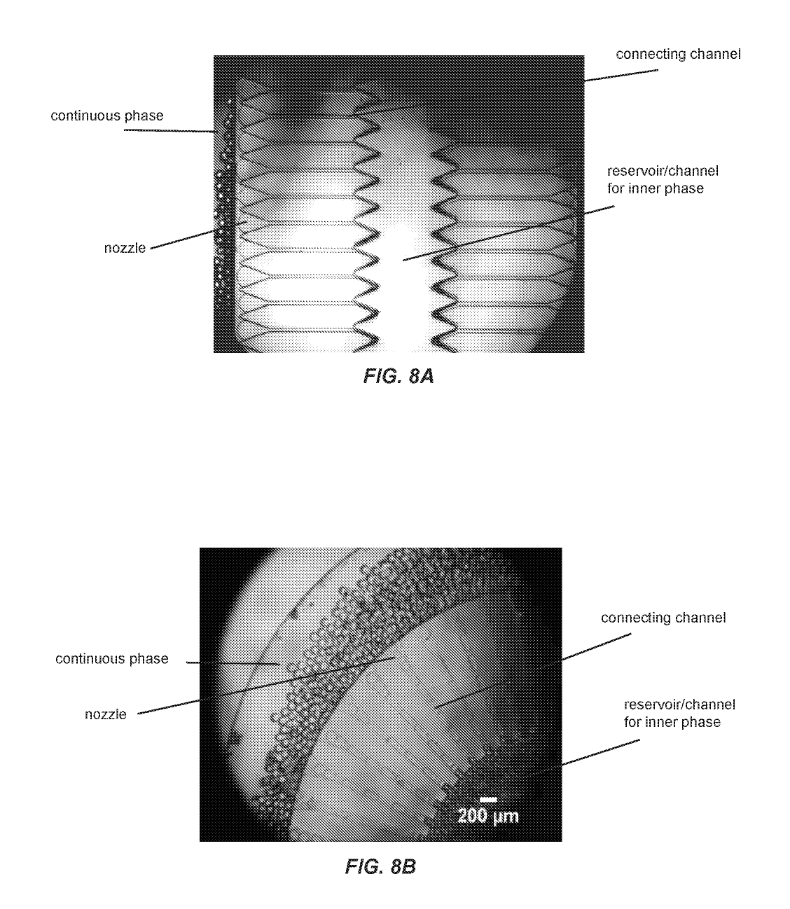

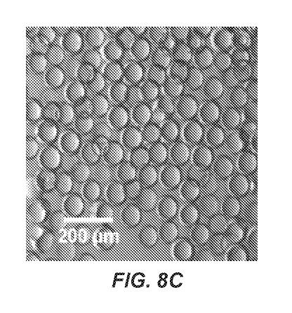

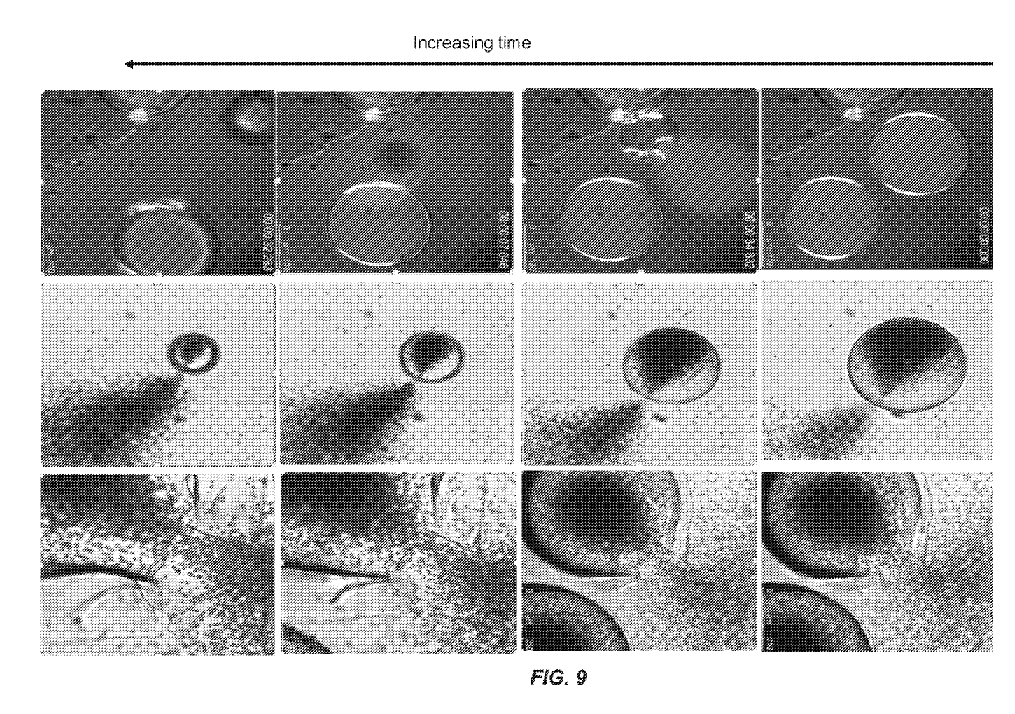

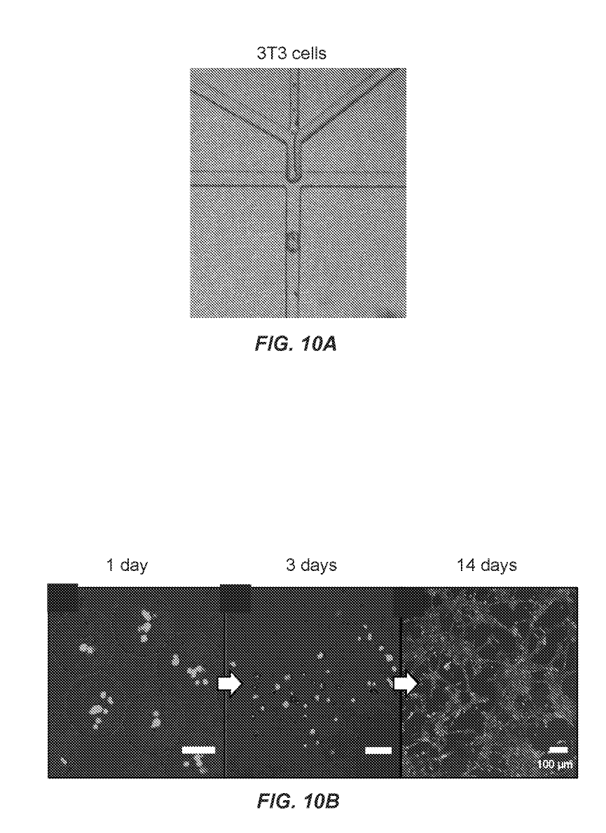

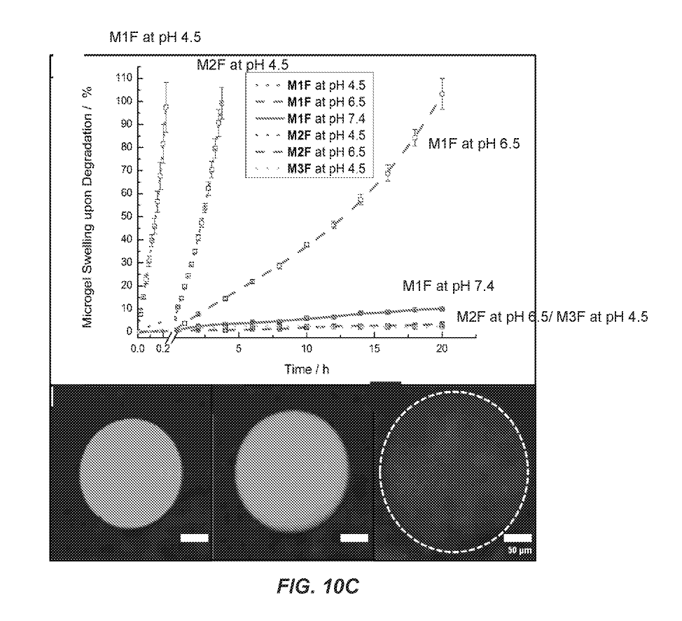

In some embodiments, microcapsules encapsulating a high viscosity solution (e.g., a high concentration active agent solution) can be formed using an exemplary process and/or microfluidic double emulsion device as shown in FIGS. 5A-5C. For example, as shown in FIG. 5A, an inner fluid comprising a high viscosity aqueous solution of interest (e.g., a high concentration active agent solution) can be introduced through the injection tube disposed at a first end of an outer tube. In some embodiments, the inner fluid can be a pre-emulsion comprising a high viscosity aqueous solution of interest (e.g., a high concentration active agent solution) dispersed in a volatile or evaporable oil phase or organic solvent. In one embodiment, the pre-emulsion was created with about 2:3 volume ratio of the aqueous phase comprising an active agent to the oil phase or organic solvent (e.g., perfluorohexane). Other volume or weight ratio that is greater than 2:3 or smaller than 2:3 of an aqueous phase comprising an active agent to an oil phase or organic solvent can also be used. For example, the volume ratio of dispersed aqueous phase comprising an active agent to an oil phase or an organic solvent can range from about 10:1 to about 1:10. In some embodiments, the volume ratio of dispersed aqueous phase comprising an active agent to an oil phase or an organic solvent can be about 1:0.3, 1:0.4, 1:0.5, 1:0.6, 1:0.7, 1:0.8, 1:0.9, 1:1, 1:2, 1:3, 1:4, 2:3, 2:4, or 3:4.

The pre-emulsion can be formed by any art-recognized methods to make an emulsion, including, e.g., but not limited to shaking, vortex emulsification, ultrasound emulsification, spontaneous emulsification, membrane emulsification, vibrating nozzle emulsification, high pressure homogenization, mechanical homogenization, rotor stator homogenization, magnetic stirring, mechanical stirring, static mixing, using a microfluidic device, and a combination of two or more thereof.

While the pre-emulsion was flowing across the injection tube, a middle fluid was introduced into the outer tube from the first end. In one embodiment, the middle fluid comprised, consisted essentially of, or consisted of polycaprolactone (Mw: ˜3500 g/mol) in dichloromethane (˜150 g/mL). Other volatile organic solvents comprising one or more polymers can also be used as the middle fluid. Large droplets of the pre-emulsion (e.g., a ˜2:3 (v/v) ratio aqueous phase to perfluorohexane) were formed in the middle fluid as the pre-emulsion exited from the tapered end of the injection tube and contacted the middle fluid (e.g., polycaprolactone (e.g., Mw: ˜3500 g/mol) in dichloromethane (e.g., 150 g/mL) in the outer tube. As the inner and middle fluids continue to move into a collection tube disposed at an opposing end of the outer tube, where an outer fluid (e.g., ˜10% polyvinyl alcohol in water) is introduced into the outer tube from the opposing end, smaller droplets of the inner fluid with a thin shell of the middle fluid are created and dispersed in the outer fluid, forming a double emulsion flowing across the collection tube. In some embodiments, the shell (middle phase) and solvent of the inner phase can then be evaporated to yield capsules.

In one embodiment, a viscous aqueous solution of about 600 cP is encapsulated by using the process and/or device as shown in FIGS. 5A-5C, where the inner phase is a pre-formed emulsion of an aqueous phase (e.g., comprising an active agent at high concentration) and perfluorohexane in a ˜2:3 v/v ratio, the middle phase is polycaprolactone (Mw: 3500 g/mol) in dichloromethane (˜150 g/mL), and an outer phase is about 10% polyvinyl alcohol in water.

The characteristics and release kinetics of the droplets can be evaluated using any methods known in the art. For example, aggregation and/or activity tests can be performed, e.g., using size exclusion chromatography (SEC), dynamic light scattering (DLS), and/or ELISA.

### Example 3

### Dropmaking

Dropmakers: Double-emulsion glass capillary devices were made by pulling round glass capillaries having an outer diameter of 1 mm to create 30 μm tips using a Sutter Instruments Model P-97 capillary puller. The tips were then sanded down to create a 60 μm tip for the inflow tube and a 120 μm tip for the outflow tube. These capillaries were then aligned coaxially inside a square capillary having an inner diameter of 1.05 mm and all the elements were fixed with epoxy. Blunt needles were fitted on both ends of the square capillary to act as injection ports for the middle and outer phases. The inner phase was was injected into the end of the 60 μm tipped round capillary by directly connecting to PE/5 polyethylene tubing (SAI Infusion Technologies, Lake Villa, Ill.). The droplets were collected directly from the blunt end of the 120 μm tipped round capillary.

Materials: For all experiments, the middle phase consisted of 10% poly-lactide-co-glycolide (PLGA) (50:50 L:G ratio, M_{w}=38,000-54,000) or polycaprolactone (PCL) (M_{w}=45,000) in dichloromethane (DCM) and the outer phase consisted of 10% polyvinyl alcohol (PVA) (M_{w}=89,000-98,000) in water.

For experiments performed at 1 cP, the inner phase consisted of water with 5 μM fluorescein isothiocyanate and 50 μg/ml Allura Red as trackers.

For experiments performed at 10 cP, the inner phase consisted of 5% PVA in water with 5 μM fluorescein isothiocyanate and 50 μg/ml Allura Red as trackers.

For experiments performed at 80 cP, the inner phase consisted of 5% Kollidon 90F (BASF, polyvinylpyrollidone, M_{w}=1,000,000-1,500,000) in water with 5 μM fluorescein isothiocyanate and 50 μg/ml Allura Red as trackers.

For experiments performed at 200 cP, the inner phase consisted of 7.5% Kollidon 90F in water with 5 μM fluorescein isothiocyanate and 50 μg/ml Allura Red as trackers.

For experiments performed at 600 cP, the inner phase consisted of 10% Kollidon 90F in water with 5 μM fluorescein isothiocyanate and 50 μg/ml Allura Red as trackers.

The solutions were loaded into syringes and attached to the double-emulsion glass capillary device with PE/5 tubing. Flow was started using Harvard Apparatus PHD 22/2000 syringe pumps and adjusted until the formation of stable droplet occurred. Dropmaking was monitored using a Phantom V9.0 (Vision Research, Wayne, N.J.) high speed camera with recording speed of 1800 fps.

### Results

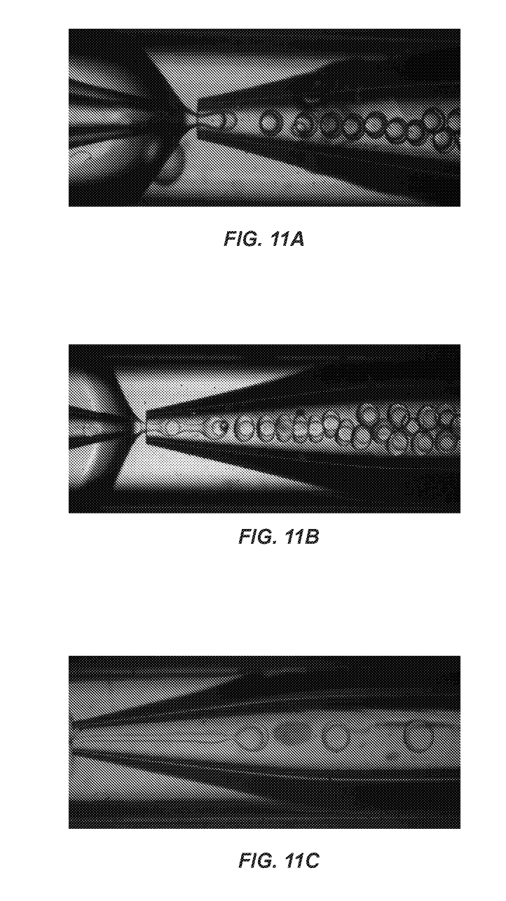

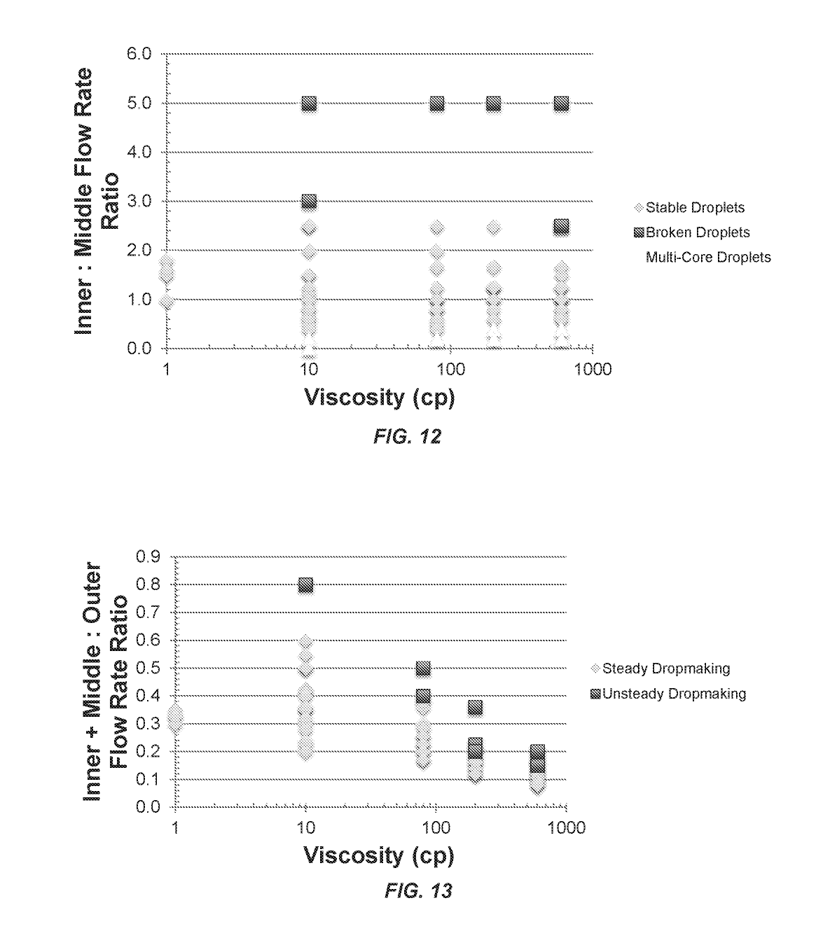

The data in FIGS. 11A-13 show that the flow rate ratios between the inner fluid and the middle fluid (I:M) and the inner fluid+middle fluid and the outer fluid (I+M+O) determine the feasibility of creating droplets for a given viscosity rather than the individual flow rates.

Inner phase:Middle phase ratios: For viscosities up to 200 cP, at Inner phase:Middle phase ratios higher than 2.5, the shell is too thin to maintain the necessary surface tension to form a double emulsion droplet. At 600 cP, this starts happening at Inner phase:Middle phase ratio of 2.5, indicating that increasing the viscosity of the inner phase beyond a certain point requires a thicker middle phase shell to make a stable droplet. Additionally, double core droplets were formed at very low Inner phase:Middle phase ratios, and this tendency slightly increased with increase in viscosity.

Inner phase+Middle phase:Outer phase ratios: This ratio determines the droplet breakup, with lower ratios being necessary as the viscosity increases. At 10 cP, droplets were formed with an Inner phase+Middle phase:Outer phase ratio of 0.6. At 80 cP, dropmaking became unsteady at 0.4, at 200 cP, it was slightly unsteady at 0.2, and completely unsteady at 0.36. At 600 cP, dropmaking was not possible at an Inner phase+Middle phase:Outer phase ratio higher than 0.167. Significant jetting was seen at ratios of 0.15 to 0.167.

Inner phase:Outer phase ratios: Inner phase:Outer phase ratio did not seem to be indicative of success in dropmaking for the experiments.

1. A method of producing an injectable composition comprising a high-concentration dose of an active agent, the method comprising:
forming an emulsion comprising droplets dispersed an injectable carrier liquid, wherein:
the droplets comprise a first liquid, the first liquid comprising an active agent at a concentration of at least about 50 mg/mL; and
the droplets and the injectable carrier liquid are substantially immiscible, thereby producing an injectable composition comprising a high-concentration dose of an active agent.

2.-5. (canceled)

6. A method of producing an injectable composition comprising a high-viscosity dose of an active agent, the method comprising:
forming an emulsion comprising droplets dispersed in an injectable carrier liquid, wherein:
the droplets comprise a first liquid, the first liquid comprising an active agent and having a viscosity of at least about 20 cP; and
the droplets and the injectable carrier liquid are substantially immiscible, thereby producing an injectable composition comprising a high-viscosity dose of an active agent.

7.-51. (canceled)

52. A vial comprising a composition comprising:
droplets comprising a first liquid, the first liquid comprising an active agent at a concentration of at least about 50 mg/mL or higher; and
a carrier liquid, wherein the carrier liquid and the droplets are substantially immiscible, and the droplets are dispersed in the carrier liquid.

53. A vial comprising a composition comprising:
droplets comprising a first liquid, the first liquid comprising an active agent and having a viscosity of at least about 20 cP or higher; and
a carrier liquid, wherein the carrier liquid and the droplets are substantially immiscible, and the droplets are dispersed in the carrier liquid.

54.-56. (canceled)

57. An injection device comprising a chamber and an injectable composition disposed in the chamber, the injectable composition comprising:
droplets comprising a first liquid, the first liquid comprising an active agent at a concentration of at least about 50 mg/mL or higher; and
an injectable carrier liquid, wherein the injectable carrier liquid and the droplets are substantially immiscible, and the droplets are dispersed in the injectable carrier liquid.

58. An injection device comprising a chamber and an injectable composition disposed in the chamber, the injectable composition comprising:
droplets comprising a first liquid, the first liquid comprising an active agent and having a viscosity of at least about 20 cP or higher; and
an injectable carrier liquid, wherein the injectable carrier liquid and the droplets are substantially immiscible, and the droplets are dispersed in the injectable carrier liquid.

59.-64. (canceled)

65. A method of administering to a subject a high concentration dose of an active agent comprising injecting the subject with an injectable composition, the injectable composition comprising:
droplets comprising a first liquid, the first liquid comprising an active agent at a concentration of at least about 50 mg/mL or higher; and
an injectable carrier liquid, wherein the injectable carrier liquid and the droplets are substantially immiscible, and the droplets are dispersed in the injectable carrier liquid.

66. A method of administering to a subject a high concentration dose of an active agent comprising injecting the subject with an injectable composition, the injectable composition comprising:
droplets comprising a first liquid, the first liquid comprising an active agent and having a viscosity of at least about 20 cP or higher; and
an injectable carrier liquid, wherein the injectable carrier liquid and the droplets are substantially immiscible, and the droplets are dispersed in the injectable carrier liquid.

67.-72. (canceled)
